(19) United States
(12) Patent Application Publication
(10) Pub. No.: US 2022/0389363 A1
(43) Pub. Date: Dec. 8, 2022

(54) Filter Apparatus and Method for Purifying Biological Processes and Cell Populations

(71) Applicant: LONZA WALKERSVILLE, Inc., Walkersville, MD (US)

(72) Inventors: Yonatan Levinson, Silver Spring, MD (US); Alex Sargent, Shady Grove, MD (US); Nicholas Uth, Washington, DC (US); Farjad Shafighi, Shady Grove, MD (US)

(21) Appl. No.: 17/770,460

(22) PCT Filed: Jan. 8, 2021
(86) PCT No.: PCT/US2021/012639

§ 371 Date: Apr. 20, 2022

Related U.S. Application Data
(60) Provisional application No. 63/109,991, filed on Nov. 5, 2020.
(60) Provisional application No. 62/959,575, filed on Jan. 10, 2020.
(60) Provisional application No. 62/959,555, filed on Jan. 10, 2020.
(60) Provisional application No. 62/959,511, filed on Jan. 10, 2020.

(51) Int. Cl. C12M 1/26; C12N 5/0783

(52) U.S. Cl. CPC C12M 33/14; C12N 5/0636

(57) ABSTRACT

A filter apparatus is disclosed for withdrawing a fluid medium from a bioreactor during the growth of a cell culture within the bioreactor. Also disclosed is a method for culturing cells in a bioreactor. The filter apparatus includes a hollow tubular member attached to a filter member. The filter member has a pore size and volume capable of withdrawing a fluid medium at a relatively high flow rate from the bioreactor. Without withdrawing biological cells from the bioreactor and without damaging or harming the cells. The filter apparatus of the present disclosure allows for many process improvements.

### BACKGROUND

Bioreactors, which are apparatuses in which biological reactions or processes can be carried out on a laboratory or industrial scale, are used widely within the biopharmaceutical industry. Bioreactors can be used in batch applications, where biological materials supplied to a bioreactor remain in the bioreactor until the end of the reaction time. Alternatively, bioreactors can be used in perfusion applications, wherein the fluid medium contained within the bioreactor is periodically or continuously removed and resupplied to the bioreactor in order to replenish nutrients contained within the fluid medium and for possibly removing damaging by-products that are produced during the process

Bioreactors, for instance, are used to produce biologics which are biological drugs that are produced from living organisms. Bioreactors are also used in immunotherapy, which is a type of treatment that boosters a patient's immune system for fighting cancer, infections, and other diseases. Immunotherapy processes, for instance, can include the production of T-cells and/or natural Natural Killer (NK) cells. During T-cell therapy, for instance, T-cells are removed from a patient's blood. The T-cells are then sent to a bioreactor and expanded or cultivated. In addition, the T-cells can be changed so that they have specific proteins called receptors. The receptors on the T-cells are designed to recognize and target unwanted cells in the body, such as cancer cells. The modified T-cells are cultivated in a bioreactor to achieve a certain cell density and then supplied to a patient's body for fighting cancer or other diseases. T-cell therapy is typically referred to chimeric antigen receptor (CAR) T-cell therapy. The use of T-cells for CAR therapy has recently proliferated due to great success in combating blood diseases.

Similarly, NK cells can be cultivated and expanded in bioreactors for infusion into a patient's body. NK cells are a type of cytotoxic lymphocyte that can seek out and destroy infected cells within the body. NK cells can display very fast immune reaction responses. Consequently, the use of NK cells in anticancer therapy has grown tremendously in interest and popularity. There is only a limited number of NK cells in the blood of a mammal, however, requiring that NK cells be grown to relatively high cell densities within bioreactors.

During the expansion of T-cells, NK cells, or other mammalian cells for producing biologics, the regulation of key metabolites in the fluid medium of the bioreactor can have a direct impact on the quality of the product that is produced. For example, during cell growth and viability, nutrient levels, lactate concentration, dissolved oxygen, pH and the like should be carefully controlled and monitored.

In addition to maintaining the cells in a carefully controlled environment, the culturing of cells for human use also requires a somewhat complex process from inoculation to use in patients. For example, when producing CAR T-cells, the cells typically first are activated and then subjected to gene editing. Once the cells are edited, the cells are expanded to achieve a certain cell density. Expansion, for instance, can take greater than 15 days, such as greater than 20 days. After expansion, the cells are purified by removing unwanted biological byproducts and unusable cells. During the process, the cells also need to be washed and removed from the growth medium. This process can also take multiple cycles. Finally, the cells are combined with a buffer and administered to a patient or placed in containers for freezing.

Concentrating the therapeutic cells and transferring them from one solution into another during the process can occur at multiple stages and can also take a substantial amount of time and manual labor. Washing and separating the therapeutic cells, however, can be an important part of the overall efficacy of the cell therapy and can prevent adverse side effects of the patient.

As can be appreciated, the above processes or methods can not only take a substantial amount of time but can also require a significant amount of human handling. When dealing with cancer patients, however, time is of the essence. Thus, a need exists for a streamline process for producing therapeutic cells and biologics.

Further, many past methods where not capable of being scalable. Thus, many of the above processes occurred in small bioreactors such as volumes of less than 2 liters. Thus, a need also exists for a process and method for culturing therapeutic cells and biologics that are scalable for producing a greater quantity of product in the same amount of time.

A need also exists for processes and methods for culturing cells and/or producing biologics that can also be automated for reducing the use of manual labor. For example, a need exists for a closed system for culturing and purifying cells, such as T-cells and NK cells.

### SUMMARY

In general, the present disclosure is directed to a filter apparatus capable of removing a fluid culture medium from bioreactors without damaging the bioreactor or cells contained within the reactor. More particularly, the present disclosure is directed to a filter apparatus that is particularly designed to remove fluid mediums from bioreactors at relatively high flow rates. As will be described in greater detail below, the filter apparatus is particularly well adapted for removing fluids without removing or harming the cells contained in the bioreactor. The present disclosure is also directed to a method for promoting cell growth in a bioreactor system in which the filter apparatus is used to remove fluid medium for replenishment and further growth of the cells and/or for purification.

In one aspect, for instance, the present disclosure is directed to a method for purifying a cellular population. The method includes expanding a biological cell population in a fluid medium. The biological cell population comprises biological cells in an unsupported state, meaning that the cells are not attached to any adjacent surface. The biological cell population contained in the bioreactor has a cell density of at least 1×10⁶ cells/mL. In accordance with the present disclosure, the fluid medium is removed and filtered from the bioreactor. More particularly, the fluid medium is filtered through a filter apparatus comprising a filter member. The filter member has a pore size that inhibits the biological cells from being withdrawn from the bioreactor as the fluid medium is withdrawn. The method further includes the step of adding to the biological cell population a buffer medium to replace the withdrawn fluid medium. Through this process, the biological cell population is washed with the buffer medium while the method is also well suited to removing biological byproducts that may be present with the biological cell population. The filter member, for instance, can have a pore size that permits passage of the biological byproducts within the fluid medium that is withdrawn without also removing or harming the biological cells. The biological byproducts, for instance, can comprise proteins, serum, and mixtures thereof. For example, after the fluid medium is withdrawn from the bioreactor, the biological cell population may contain biological byproducts (or any of the above described individual byproducts) in an amount less than about 0.1% by weight.

During the above method, greater than about 40%, such as greater than about 50%, such as greater than about 60%, such as greater than about 70%, such as greater than about 80% of the volume of the fluid medium can be withdrawn and at least partially replaced with the buffer medium. The biological cell population and fluid medium, for instance, can have a volume in the bioreactor of from about 1 L to about 10 L in one embodiment, or from about 5 L to about 75 L in another embodiment. The fluid medium can be withdrawn from the bioreactor at a flow rate such that at least 50% of the volume of the fluid medium in the bioreactor is withdrawn in a period of less than about 1 hour. The filter apparatus of the present disclosure, for instance, can operate at a relatively high flow rate without blockages forming on the exterior surface of the filter apparatus.

As described above, the above method can wash the biological cell population and remove biological byproducts. The method can be repeated multiple times in order to purify the cells. For instance, the method can be repeated from about 2 cycles to about 5 cycles.

After the biological cells have been purified as described above, the biological cell population and buffer medium can be placed into flexible bag vessels for cryogenic storage. In one aspect, a cryogenic buffer medium can also be combined with the biological cell population.

All different types of biological cells can be purified according to the method as described above. For instance, the biological cells can comprise any suitable mammalian cells. The method of the present disclosure is particularly well suited to expanding and purifying T-cells and NK cells.

In some applications, the biological cell population may contain different cell types, such as first cells and second cells. One of the cell types may be particularly well suited for administration to patients, while the other cell type may have other uses or may be discarded. The method for the present disclosure provides an efficient manner for separating the first cell types from the second cell types in order to further purify the biological cell population.

For example, the biological cell population containing at least two different cell types can be placed in contact within the bioreactor with one or more microcarriers. The microcarriers can be added to the fluid medium in which the biological cell population is contained. The microcarriers can be designed such that the first cells attach and bind to the surface of the microcarriers while the second sells do not. In accordance with the present disclosure, the fluid medium within the bioreactor can be removed and filtered through a second filter apparatus. The second filter apparatus can have a pore size that permits passage of the second cells but inhibits passage of the microcarriers for separating the first cells from the second cells.

The second cells, once separated from the first cells, can then be subjected to further purification and washing steps as described above and then placed in use or stored for future use.

The first cells can also be isolated and used as desired. For example, in one embodiment, the microcarriers added to the bioreactor can be dissolvable. The microcarriers, for instance, can be dissolvable in the fluid medium or can dissolve when contacted with a dissolving agent added to the fluid medium. Once the microcarriers have dissolved, the first cells remain in the bioreactor in an unsupported state. The first cells can then be purified and washed according to the method described above and used as desired.

As described above, the methods of the present disclosure are carried out with the use of a filter apparatus. In one embodiment, the filter apparatus can include a hollow tubular member for filtering fluid from a bioreactor.

The hollow tubular member may have a length sufficient for insertion into a bioreactor. For instance, the hollow tubular member can have a length sufficient to extend towards the bottom of a bioreactor. The hollow tubular member may extend through a port in the top or side of the bioreactor. The hollow tubular member has a first end defining a first opening and a second and opposite end defining a second opening. The second opening is for insertion into a fluid medium in a bioreactor and for withdrawing the fluid medium. The second opening of the hollow tubular member can have a cross-sectional area designed to be capable of withdrawing a desired volumetric flow rate from the bioreactor.

In accordance with the present disclosure, the filter apparatus further includes a filter member located at the second end of the hollow tubular member. The filter member can completely surround and enclose the second opening. The filter member defines an interior surface and an exterior surface. The filter member comprises a porous material. In accordance with the present disclosure, the porous material has an absolute pore size of from about 1 micron to about 9 microns, such as from about 1 micron to about 6 microns. The above pore sizes have been found to allow fluid medium to be withdrawn from the bioreactor without removing the biological cells. In one aspect, the filter member comprises a porous mesh. In another aspect, the filter member comprises a nonwoven mesh formed from sintered metal fiber. The sintered metal fiber, for instance, may comprise stainless steel. Alternatively, the filter member (and hollow tubular member) may also be made from a polymer material. For example, the filter member can be made from a polymer mesh or a nonwoven. The polymer material can comprise, for instance, a polyamide or a polyolefin.

The filter member of the filter apparatus can have sufficient surface area to allow for relatively high volumetric fluid flow rates. For instance, the surface area can be greater than about 0.5 in². The surface area of the exterior surface, for instance, can be greater than about 3 in², such as greater than about 4 in², such as greater than about 6 in², and generally less than about 50 in². The length of the filter member can depend upon various factors. In one aspect, the filter member can have a length along an axial direction of the hollow tubular member of from about 2 inches to about 8 inches.

The hollow tubular member and the second opening can generally have a diameter of greater than about 2 mm, such as greater than about 4 mm, such as greater than about 8 mm, such as greater than about 10 mm, such as greater than about 12 mm, such as greater than about 14 mm, such as greater than about 16 mm, such as greater than about 18 mm, such as greater than about 20 mm. The diameter of the hollow tubular member is generally less than about 50 mm, such as less than about 30 mm, such as less than about 20 mm, such as less than about 14 mm.

The ratio between the cross-sectional area of the second opening and the surface area of the filter member can generally be from about 1:5 to about 1:200, such as from about 1:15 to about 1:100.

In one aspect, the interior surface of the filter member can have a different absolute pore size than the exterior surface of the filter member. For example, the interior surface of the filter member can have a pore size that is larger than the pore size of the exterior surface. The pore size of the exterior surface, for example, can be from about 1 micron to about 9 microns while the absolute pore size of the interior surface of the filter member can be from about 5 microns to about 20 microns.

As described above, in one embodiment, a second type of filter apparatus can be used that permits passage of a first type of cell while preventing passage of a second type of cell that maybe bound to a microcarrier. In this application, the filter apparatus can be as described above but can have a larger pore size. For instance, the filter member of the filter apparatus can have an absolute pore size of greater than about 60 microns, such as greater than about 70 microns, such as greater than about 80 microns, such as greater than about 90 microns, and generally less than about 150 microns, such as less than about 130 microns, such as less than about 120 microns, such as less than about 110 microns.

Furthermore, in one aspect, the present disclosure is generally directed to a filter apparatus that includes a hollow tubular member for filtering fluid from a bioreactor. The hollow tubular member may have a length sufficient for insertion into a bioreactor. For instance, the hollow tubular member can have a length sufficient to extend towards the bottom of a bioreactor. The hollow tubular member may extend through a port in the top or side of the bioreactor. The hollow tubular member has a first end defining a first opening and a second and opposite end defining a second opening. The second opening is for insertion into a fluid medium in a bioreactor and for withdrawing the fluid medium. The second opening of the hollow tubular member can have a cross-sectional area designed to be capable of withdrawing a desired volumetric flow rate from the bioreactor.

The filter member of the filter apparatus can have sufficient surface area to allow for relatively high volumetric fluid flow rates. For instance, the surface area can be greater than about 0.5 in². The surface area of the exterior surface, for instance, can be greater than about 3 in², such as greater than about 4 in², such as greater than about 6 in², and generally less than about 50 in². The length of the filter member can depend upon various factors. In one aspect, the filter member can have a length along an axial direction of the hollow tubular member of from about 2 inches to about 8 inches.

In one embodiment, the hollow tubular member is straight from the first end to the second end. In an alternative embodiment, the hollow tubular structure can have a shape such that the second end does not interfere with an impeller that can be rotating in the bioreactor. For example, in one embodiment, the hollow tubular member can include a first straight section, a second straight section, and an angular section positioned between the first straight section and the second straight second. The angled section can extend from the first straight section at an angle of from about 25° to about 45°. Similarly, the angled section can extend from the second straight section at an angle of from about 25° to about 45°. In one embodiment, the first straight section and the second straight section are parallel to a vertical axis that extends through the bioreactor.

In one embodiment, the hollow tubular member can also include an angular member located at the second end. The hollow tubular member can include a straight member that transitions into the angular member. The angular member can be at an angle to the straight section of from about 50° to about 90°. For example, in one embodiment, the angular member forms a right angle at the end of the hollow tubular member. In this regard, when the filter apparatus is extended into a bioreactor, the angular member can be positioned towards the bottom of the bioreactor and can be generally parallel with the bottom surface of the bioreactor. For instance, in one embodiment, the angular member can be designed to place the filter member below an impeller contained within the bioreactor.

In one embodiment, the hollow tubular member and the filter member can be completely enclosed for sterile closed connection to a port of the bioreactor. A plastic, flexible bellows can enclose the hollow tubular member and the filter member. A sterile connection port may be attached to one end of the bellows. The bioreactor port may have a matching sterile connector. When the matching sterile connectors of the bioreactor and the bellows are connected, the bellows may be collapsed and the filter member and hollow tubular member may be inserted into the bioreactor port.

In one embodiment, the filter apparatus can include a filter member on a side or bottom wall of the bioreactor. The filter member can be a mesh patch on the side or bottom wall of the bioreactor. A flexible cone can connect the mesh patch to a hollow tubular member for output of fluid from the bioreactor.

The present disclosure is also directed to a method for culturing cell growth. The method includes inoculating biological cells into a bioreactor, such as T-cells or NK cells. The bioreactor contains a fluid medium for cell growth. The fluid medium is perfused by inserting into the bioreactor a filter apparatus as described above. The filter member of the filter apparatus can have a pore size that inhibits the biological cells from being withdrawn from the bioreactor as the fluid medium is withdrawn. The method further includes the step of replenishing the fluid medium within the bioreactor in order to promote cell viability.

In one embodiment, for instance, the filter apparatus can be designed to remove the fluid medium at a rate of greater than about 0.5 L per day, such as greater than about 1 L per day, such as greater than about 2 L per day, such as greater than about 5 L per day, such as greater than about 10 L per day, such as greater than about 15 L per day, such as greater than about 25 L per day.

In accordance with the present disclosure, the filter apparatus further includes a filter member located at the second end of the hollow tubular member. The filter member can completely surround and enclose the second opening. The filter member defines an interior surface and an exterior surface. The filter member comprises a porous material. In accordance with the present disclosure, the porous material has an absolute pore size of from about 1 micron to about 9 microns, such as from about 1 micron to about 6 microns. In one aspect, the filter member comprises a porous mesh. In another aspect, the filter member comprises a nonwoven mesh formed from sintered metal fiber. The sintered metal fiber, for instance, may comprise stainless steel. Alternatively, the filter member (and hollow tubular member) may also be made from a polymer material. For example, the filter member can be made from a polymer mesh or a nonwoven. The polymer material can comprise, for instance, a polyamide or a polyolefin.

The present disclosure is also directed to a method for culturing cells. The method includes inoculating biological cells into a bioreactor. The bioreactor comprises a stirred tank bioreactor. The bioreactor contains a fluid medium for cell growth that is agitated during cell growth. The biological cells are present in the bioreactor in an unsupported state. In accordance with the present disclosure, the fluid medium is perfused from the bioreactor through a filter apparatus that is in contact with the fluid medium within the bioreactor. The filter apparatus comprises a filter member having a pore size that inhibits the biological cells from being withdrawn from the bioreactor as the fluid medium is withdrawn. The fluid medium within the bioreactor is replenished as fluid medium is withdrawn from the bioreactor in order to promote cell viability.

The biological cells that are contained within the bioreactor can comprise any suitable cells, such as any suitable mammalian cells. In certain aspects, the biological cells are T-cells or NK cells. The initial cell density after inoculation is generally less than about 0.5×10⁶ cells/mL, such as less than about 0.4×10⁶ cells/mL, such as less than about 0.3×10⁶ cells/mL. The initial cell density is generally greater than about 0.1×10⁴ cells/mL.

In one aspect, perfusion is initiated after the biological cells in the bioreactor have reached a desired cell density. For instance, perfusion can be initiated after the biological cells reach a cell density of greater than about 1×10⁶ cells/mL, such as greater than about 1.5×10⁶ cells/mL, such as greater than about 1.7×10⁶ cells/mL. For example, perfusion can be initiated after a period of at least 4 days, such as at least 7 days after the bioreactor has been inoculated with the biological cells.

The biological cells can be rapidly and dramatically expanded during the process. For instance, the biological cells within the bioreactor can reach a cell density of at least about 1×10⁷ cells/mL, such as at least 1.2×10⁷ cells/mL. The cells can be harvested from the bioreactor after about 14 days, such as after about 13 days.

During the process, various parameter levels can be controlled due to the manner in which the bioreactor is operated. For instance, glucose levels within the fluid medium can stay above 4 g/L, such as greater than about 5 g/L. Lactate levels, on the other hand, can remain below about 1.5 g/L, such as below about 1.3 g/L during the entire process. In one aspect, dissolved oxygen during the process within the fluid medium rapidly decreases to 0 or almost 0. It is believed that the reduction in dissolved oxygen can increase a desired phenotype. For instance, the process can be operated so that there is a proportionate increase in a phenotype that is particularly desired. The phenotype, for instance, can comprise T_{scm} cells.

During perfusion, the fluid medium is withdrawn from the bioreactor using a filter apparatus having a filter member with an absolute pore size of from about 1 micron to about 9 microns, such as from about 1 micron to about 6 microns. The filter member can have a surface area of generally greater than about 0.5 in², such as greater than about 2 in², and generally less than about 10 in². During perfusion, at least about 30%, such as at least about 40%, such as least about 50%, such as at least about 60%, such as at least about 70% of the volume of the fluid medium within the bioreactor is perfused every 24 hours and replaced.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a cross-sectional view of one embodiment of a bioreactor system in accordance with the present disclosure;

FIG. 2 is a side view of one embodiment of a filter apparatus made in accordance with the present disclosure;

FIG. 3 is a side view of another embodiment of a filter apparatus made in accordance with the present disclosure;

FIG. 4A is a perspective view of one embodiment of a filter member attached to a filter apparatus in accordance with the present disclosure;

FIG. 4B is a side view of the filter member illustrated in FIG. 4A;

FIG. 4C is another side view of the filter member illustrated in FIG. 4A;

FIG. 5A is a side view of another embodiment of a filter apparatus made in accordance with the present disclosure;

FIG. 5B is a partial side view of the filter apparatus illustrated in FIG. 5A;

FIG. 6 is a perspective view of another embodiment of a bioreactor system in accordance with the present disclosure;

FIGS. 7A through 7C are perspective views of one embodiment of bioreactor system having a sterile closed connection between the bioreactor and the filter apparatus;

FIG. 8A is a perspective view of another embodiment of a bioreactor system in accordance with the present disclosure;

FIG. 8B is a side view of a filter apparatus that may be used with the bioreactor system illustrated in FIG. 8A;

FIG. 9 is a side view of another embodiment of a bioreactor system in accordance with the present disclosure;

FIG. 10 is a side view of another embodiment of a filter apparatus that may be used in accordance with the present disclosure;

FIGS. 11-17 are graphical representations of results obtained in the examples described below;

FIG. 18 is a cross-sectional view of a bioreactor illustrating a method according to the present disclosure for separating different types of cells;

FIGS. 19A and 19B are a representation of results obtained according to the present disclosure before and after using magnetic beads as microcarriers;

FIGS. 19C, 19D, and 19E are a representation of results obtained according to the present disclosure before and after using magnetic beads as microcarriers according to FIGS. 19A and 19B;

FIGS. 20A-20C are a representation of results obtained according to the present disclosure using magnetic beads as microcarriers in the presence of a magnet;

FIGS. 20D-20E are a representation of results obtained according to the present disclosure using magnetic beads as microcarriers 24 hours after removal of the magnet of FIGS. 20A-20C; and

FIGS. 21A-B illustrate a cross-sectional view of a bioreactor having a skimmer filter according to the present disclosure.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to methods and systems for cultivating and propagating cells and/or cell products in a bioreactor. The bioreactor contains a biological cell population in a fluid medium, such as a fluid growth medium. The biological cells are cultivated under suitable conditions and in a suitable culture medium for promoting cell reproduction and growth until a desired amount of cells can be harvested from the bioreactor.

In accordance with the present disclosure, the bioreactor is designed to be run in the perfusion mode during cell culturing processes. In particular, at selected times, the fluid medium contained within the bioreactor is continuously or at least periodically removed and replenished. In the past, problems have been experienced in removing liquid mediums from bioreactors without significantly harming or damaging the cells contained in the bioreactor. Thus, in the past, bioreactors were typically operated in batch mode under static conditions or in a rocking type bioreactor. These systems have been found to be extremely inefficient in expanding cell populations. Prior systems are also not scalable and thus only operated in small bioreactor volumes.

The present disclosure is directed to an improved bioreactor system that includes a stirred tank bioreactor in combination with a filter apparatus. In accordance with the present disclosure, the filter apparatus is capable of rapidly removing fluid medium from the bioreactor without also removing the biological cell population, without damaging the cells, and/or without problems associated with fouling. The stirred tank bioreactor in combination with the filter apparatus of the present disclosure can provide numerous benefits and advantages. For example, through the system and process of the present disclosure, cell cultures, particularly T-cell cultures and NK cell cultures, can be rapidly and efficiently expanded in comparison to past bioreactor systems. For example, the process of the present disclosure can reach cell densities and particularly viable cell densities that were not possible with past equipment and protocols. Further, the process and system of the present disclosure also allow for carefully controlling parameters and metabolites within the bioreactor, which further promotes cell growth and the health and viability of the cells. For instance, the capability of maintaining the cells in an unsupported state (i.e. not supported on a microcarrier) in a stirred tank reactor and with the capability of operating in perfusion mode allows for careful control of lactate levels, nutrient levels including glucose levels, control of ammonia levels, in addition to controlling pH, dissolved oxygen and other parameters. Of particular advantage, all of the above parameters can be carefully controlled and monitored in a closed system that eliminates manual manipulation of the cells or the fluid medium in which the cells are maintained.

The process and system of the present disclosure have also been found to provide various other advantages and benefits in comparison to systems used in the past. For example, the process and system of the present disclosure is completely scalable and similar results can be achieved not only in smaller bioreactors but also in much larger bioreactors. For example, bioreactors incorporated into the process can have volumes greater than 1 liter, such as greater than 3 liters, such as greater than 5 liters, such as greater than 10 liters, such as greater than 15 liters, such as greater than 20 liters, such as greater than 30 liters, such as greater than 40 liters, and even greater than 50 liters. By being completely scalable, allogeneic cell therapy processes can be carried out for producing much greater amounts of product for administration to many patients in taking just a fraction of the time needed with past processes.

Initiation of cell growth is also much simpler and automated with respect to use of the present system and process. For instance, the present system and process eliminates 2D activation/seed train that was necessary in past bioreactor systems, such as in rocker-type bioreactors. Instead, biological cell populations cultivated in the present system can be activated/expanded directly.

However, in one aspect, biological cells may still be subject to 2D seed train activation and/or expansion prior to use in a system or process according to the present disclosure. in one aspect, the biological cells can be thawed into a 2D seed train flask. The biological cells may be inoculated at a seed density of about 0.1×10⁶ cells/cm² to about 1.5×10⁶ cells/cm², such as about 0.25×10⁶ cells/cm² to about 1.25×10⁶ cells/cm², such as about 0.5×10⁶ cells/cm² to about 1×10⁶ cells/cm², or any ranges or values therebetween.

As used herein, a nutrient media or matrix refers to any fluid, compound, molecule, or substance that can increase the mass of a bioproduct, such as anything that may be used by an organism to live, grow or otherwise add biomass. For example, a nutrient feed can include a gas, such as oxygen or carbon dioxide that is used for respiration or any type of metabolism. Other nutrient media can include carbohydrate sources. Carbohydrate sources include complex sugars and simple sugars, such as glucose, maltose, fructose, galactose, and mixtures thereof. A nutrient media can also include an amino acid. The amino acid may comprise, glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., 4-hydroxyproline, ε-N,N,N-trimethyllysine, 3-methylhistidine, 5-hydroxylysine, O-phosphoserine, γ-carboxyglutamate, γ-N-acetyllysine, ω-N-methylarginine, N-acetylserine, N,N,N-trimethylalanine, N-formylmethionine, γ-aminobutyric acid, histamine, dopamine, thyroxine, citrulline, ornithine, β-cyanoalanine, homocysteine, azaserine, and S-adenosylmethionine. In some embodiments, the amino acid is glutamate, glutamine, lysine, tyrosine or valine.

The nutrient media can also contain one or more vitamins. Vitamins that may be contained in the nutrient media include group B vitamins, such as B12. Other vitamins include vitamin A, vitamin E, riboflavin, thiamine, biotin, and mixtures thereof. The nutrient media can also contain one or more fatty acids and one or more lipids. For example, a nutrient media feed may include cholesterol, steroids, and mixtures thereof. A nutrient media may also supply proteins and peptides to the bioreactor. Proteins and peptides include, for instance, albumin, transferrin, fibronectin, fetuin, and mixtures thereof. A growth medium within the present disclosure may also include growth factors and growth inhibitors, trace elements, inorganic salts, hydrolysates, and mixtures thereof. Trace elements that may be included in the growth medium include trace metals. Examples of trace metals include cobalt, nickel, and the like. For instance, and for example only, in one aspect, the nutrient medium/matrix may be X-Vivo™ matrix medium sold by Lonza.

Nonetheless, the thawed biological cells are seeded for growth in the 2D seed train flask with nutrient media and maintained in the 2D seed train flask until the biological cells reach a cell density of about 2 million cells/mL or greater for T-cells. For instance, in one aspect, the biological cells may be maintained in the 2D seed train flask for about 3 days to about 9 days, such as about 4 days to about 8 days, such as about 5 days to about 7 days, in order to achieve the desired cell density.

After the desired cell density is obtained, the biological cells contained in the 2D seed train may be harvested using a passaging solution. Regardless of the passaging solution selected, the harvested biological cells may be used to inoculate a process or system described herein.

Nonetheless, as discussed, in on aspect, 2D seed train activation and/or expansion is not used, and instead, cells are activated and/or expanded using the present system and/or process.

When expanding T-cells or NK cells populations, the process and system of the present disclosure can also facilitate many downstream processes after a desired cell density is reached. For example, the filter apparatus of the present disclosure can greatly improve the efficiently of washing the cells, separating byproducts from the cells, purifying the cells, and/or transferring the cells to cryogenic storage containers. Thus, the system and process of the present disclosure is particularly well suited to expanding cell therapy populations, such as T-cell populations and NK cell populations. The system and process can be used not only to promote the growth of autologous cell therapies, but also allogeneic cell therapies. With respect to autologous cell therapies, the system and process of the present disclosure can dramatically reduce the amount of time needed to reach a desired viable cell density.

When producing allogeneic CAR T-cells or NK cells, the process and system of the present disclosure can provide immediate off the shelf cell therapies to many patients while significantly lowering costs. The process and system is also particularly efficient and well suited to removing unwanted biological by products, such as proteins, serum, and T-cell receptors from the final cell culture, and for greatly improving purity of the final product, which immediately translates into not only better patient care but also the ability to store the product in greater cell densities. Similar benefits and advantages are also achieved when producing virus specific T-cells and CAR NK cells.

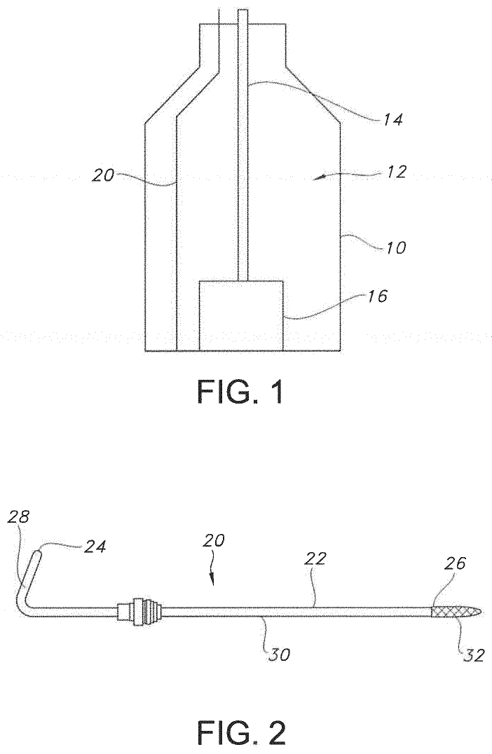

Referring to FIG. 1, one embodiment of a bioreactor system in accordance with the present disclosure is shown. The bioreactor system includes a bioreactor 10. The bioreactor 10 comprises a hollow vessel or container that includes a bioreactor volume 12 for receiving a cell culture suspended within a fluid growth medium. In accordance with the present disclosure, the biological cells contained in the biological 10 can be suspended in the fluid growth medium in an unsupported state meaning that the cells are not attached to any adjacent surfaces, such as microcarriers. The ability to process cells in an unsupported state is believed to increase the rate of expansion of the cell culture and allow for the system to be scalable. As shown in FIG. 1, the bioreactor system can further include a rotatable shaft 14 coupled to an agitator such as an impeller 16.

The bioreactor 10 can be made from various different materials. In one embodiment, for instance, the bioreactor 10 can be made from metal, such as stainless steel. Metal bioreactors are typically designed to be reused.

Alternatively, the bioreactor 10 may comprise a single use bioreactor made from a flexible polymer film. The film or shape conforming material can be liquid impermeable and can have an interior hydrophilic surface. In one embodiment, the bioreactor 10 can be made from a flexible polymer film that is designed to be inserted into a rigid structure, such as a metal container for assuming a desired shape. Polymers that may be used to make the flexible polymer film include polyolefin polymers, such as polypropylene and polyethylene. Alternatively, the flexible polymer film can be made from a polyamide. In still another embodiment, the flexible polymer film can be formed from multiple layers of different polymer materials. In one embodiment, the flexible polymer film can be gamma irradiated.

Because the process is scalable, the bioreactor 10 can have any suitable volume. For instance, the volume of the bioreactor 10 can be from 100 mL to about 10,000 L or larger. For example, the volume 12 of the bioreactor 10 can be greater than about 0.5 L, such as greater than about 1 L, such as greater than about 2 L, such as greater than about 3 L, such as greater than about 4 L, such as greater than about 5 L, such as greater than about 6 L, such as greater than about 7 L, such as greater than about 8 L, such as greater than about 10 L, such as greater than about 12 L, such as greater than about 15 L, such as greater than about 20 L, such as greater than about 25 L, such as greater than about 30 L, such as greater than about 35 L, such as greater than about 40 L, such as greater than about 45 L. The volume of the bioreactor 10 is generally less than about 20,000 L, such as less than about 15,000 L, such as less than about 10,000 L, such as less than about 5,000 L, such as less than about 1,000 L, such as less than about 800 L, such as less than about 600 L, such as less than about 400 L, such as less than about 200 L, such as less than about 100 L, such as less than about 50 L, such as less than about 40 L, such as less than about 30 L, such as less than about 20 L, such as less than about 10 L. In one embodiment, for instance, the volume of the bioreactor can be from about 1 L to about 5 L. In an alternative embodiment, the volume of the bioreactor can be from about 25 L to about 75 L. In still another embodiment, the volume of the bioreactor can be from about 1,000 L to about 5,000 L.

In addition to the impeller 16, the bioreactor 10 can include various additional equipment, such as baffles, spargers, gas supplies, ports, and the like which allow for the cultivation and propagation of biological cells. In addition, the bioreactor system can include various probes for measuring and monitoring pressure, foam, pH, dissolved oxygen, dissolved carbon dioxide, and the like.

In one embodiment, the bioreactor 10 includes a top that defines a plurality of ports. The ports can allow supply lines and feed lines into and out of the bioreactor 12 for adding and removing fluids and other materials. In addition, the bioreactor system can be placed in association with a load cell for measuring the mass of the culture within the bioreactor 10.

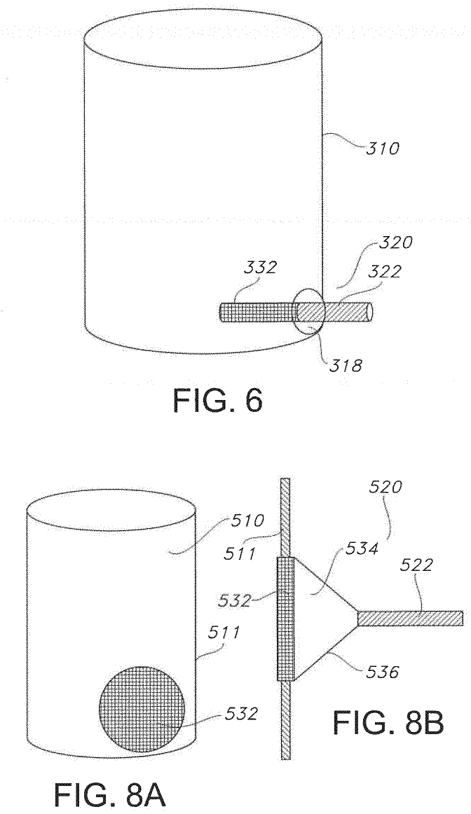

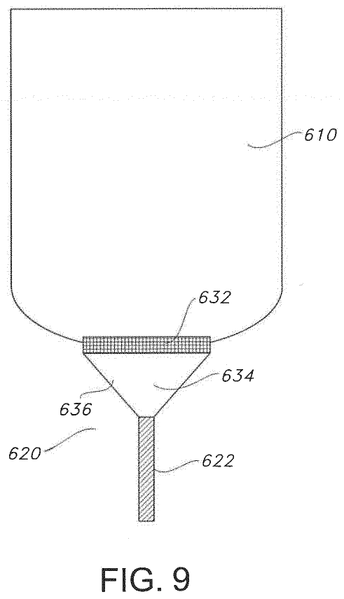

In an alternative embodiment, the plurality of ports can be located at different locations on the bioreactor 10. For instance, in one embodiment, the ports can be located on a side wall of the bioreactor, as shown in FIGS. 6-8. In another embodiment, the ports can be located at the bottom of the bioreactor, as shown in FIG. 9. For example, a bioreactor made from a flexible polymer film may include ports located on the bottom of the vessel.

As shown in FIG. 1, the bioreactor 10 can include a rotatable shaft 14 attached to at least one impeller 16. The rotatable shaft 14 can be coupled to a motor for rotating the shaft 14 and the impeller 16. The impeller 16 can be made from any suitable material, such as a metal or a biocompatible polymer. Examples of impellers suitable for use in the bioreactor system include hydrofoil impellers, high-solidity pitch-blade impellers, high-solidity hydrofoil impellers, Rushton impellers, pitched-blade impellers, gentle marine-blade impellers, and the like. In addition, the rotatable shaft 14 can be coupled to a single impeller 16 as shown in FIG. 1 or can be coupled to two or more impellers. When containing two or more impellers, the impellers can be spaced apart along the rotating shaft 14. In one embodiment, the impeller 16 is rotated an amount sufficient to maintain the biological cells contained in the bioreactor 10 in suspension in a fluid medium without damaging the biological cells.

The amount of energy imparted to the fluid medium by the one or more agitators can have an impact upon cell viability and cell growth. In one aspect, it was discovered that optimum conditions within the bioreactor are maintained when the propeller 16 is rotated at a rate of about 35 rpm or greater, such as about 40 rpm or greater, such as about 50 rpm or greater, such as about 60 rpm or greater, such as about 70 rpm or greater and generally at a rate of less than about 200 rpm, such as at a rate of less than about 120, such as at a rate of less than about 100 rpm. The tip speed of the impeller, for instance, can be greater than about 0.07 m/s, such as greater than about 0.09 m/s, such as greater than about 0.1 m/s, such as greater than about 0.12 m/s, and generally less than about 0.4 m/s, such as less than about 0.3 m/s. In terms of power input to a 1 L stirred tank bioreactor, this equates to a power input greater than 0.0012 Watts, and generally less than 0.0143 Watts, such as less than about 0.006 Watts.

In one embodiment, the bioreactor system can also include a controller which may comprise one or more programmable devices or microprocessors. The controller can be used to maintain optimum conditions within the bioreactor 10 for promoting cell growth. The controller, for instance, can be in communication and control thermal circulators, load cells, control pumps, and receive information from various sensors and probes. For instance, the controller may control and/or monitor the pH, dissolved oxygen tension, dissolved carbon dioxide, the temperature, the agitation conditions, alkali condition, fluid growth medium condition, pressure, foam levels, and the like. For example, based upon pH readings, the controller may be configured to regulate pH levels by adding requisite amounts of acid or alkali. The controller may also use a carbon dioxide gas supply to decrease pH. Similarity, the controller can receive temperature information and control fluids being fed to a water jacket surrounding the bioreactor for increasing or decreasing temperature.

In one aspect, various parameters contained within the bioreactor may be monitored using Raman spectroscopy. A Raman spectroscopy device, for instance, can measure a biomass concentration and/or various other parameters contained within the bioreactor. This information can then be fed to the controller which can make automatic adjustments to feed rates and withdrawal rates from the bioreactor for maintaining various parameters within controlled limits. The use of Raman spectroscopy in monitoring cell cultures, for example, is described in U.S. Patent Publication No. 2019/0137338, which is incorporated herein by reference.

In accordance with the present disclosure, the bioreactor 10 can also be in communication with one or a plurality of filter apparatuses 20 as shown in FIG. 1. The filter apparatus 20 can extend through a port within the top of the bioreactor 10. As shown, the filter apparatus 20 can extend into the bioreactor 10 and be placed adjacent to the bottom of the bioreactor without interfering with the impeller 16. The filter apparatus 20 is for continuously or periodically withdrawing liquid medium from the bioreactor 10 without withdrawing biological cells contained within the bioreactor. The filter apparatus 20 of the present disclosure, for instance, can withdraw fluid at a relatively high flow rate without also removing or damaging cells.

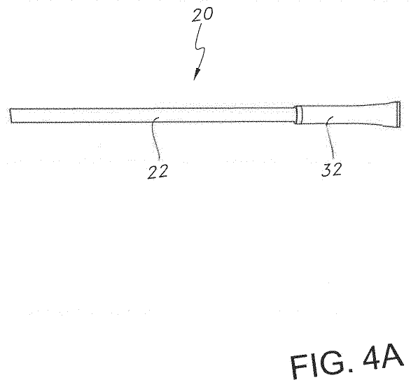

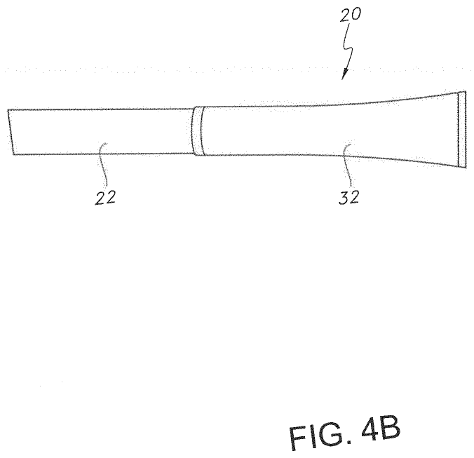

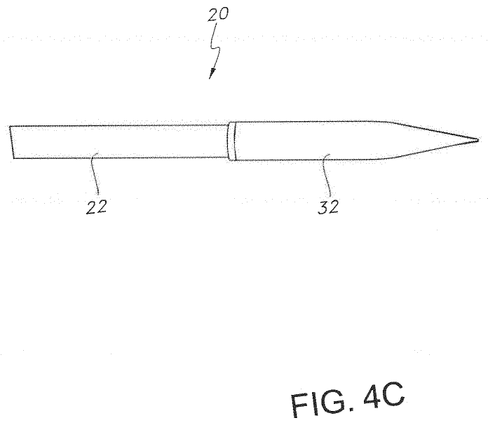

Referring to FIGS. 2, 4A, 4B and 4C, one embodiment of a filter apparatus 20 that may be used in accordance with the present disclosure is shown. Referring to FIG. 2, the filter apparatus 20 includes a hollow tubular member 22. The hollow tubular member 22 can include a first end 24 that defines a first opening and a second and opposite end 26 that defines a second opening. The hollow tubular member 22 can be made from any suitable material that is biologically compatible with cell cultures. For example, the hollow tubular member 22 can be made from a metal, such as stainless steel.

In an alternative embodiment, the hollow tubular member can be made from a polymer. In one embodiment, for instance, the filter apparatus 20 can be designed to be discarded after a single use. In this embodiment, the hollow tubular member 22 can be made from a polymer material. For instance, the hollow tubular member can be made from a polyolefin, such as polypropylene or polyethylene. Alternatively, the hollow tubular member 22 can be made from a polyamide. Otherwise, the hollow tubular member 22 can be made from a plastic material that can be gamma irradiated.

The hollow tubular member 22 can be flexible or rigid. The hollow tubular member 22, the first opening, and the second opening can generally have a diameter sized for the particular application and the amount of fluid needed to be withdrawn from the bioreactor 10. For instance, the diameter of the hollow tubular member 22 can generally be greater than about 2 mm, such as greater than about 4 mm, such as greater than about 6 mm, such as greater than about 8 mm, such as greater than about 10 mm. The diameter of the hollow tubular member 22 is generally less than about 60 mm, such as less than about 40 mm, such as less than about 20 mm, such as less than about 15 mm, such as less than about 11 mm, such as less than about 10 mm, such as less than about 8 mm.

The first end 24 of the hollow tubular member 22 can include a tubing connection for connecting the hollow tubular member 22 to plastic tubing. The tubing connection can be any of various weldable tubing types. The outer diameter of the tubing connection of the first end 24 can generally have an outer diameter sized for the particular application and the amount of fluid needed to be withdrawn from the bioreactor. For instance, the outer diameter of the tubing connection can generally be about 3 mm or more, such as about 6 mm or more, such as about 13 mm or more, such as about 19 mm or more, such as about 26 mm. The outer diameter of the tubing connection is generally about 26 mm or less.

The hollow tubular member 22 can be made from a single piece of material or can be made from multiple pieces connected together. The hollow tubular member 22 can be straight from the first end 24 to the second end 26. Alternatively, the hollow tubular member 22 can include an angular member 28 as shown in FIG. 2. In the embodiment illustrated in FIG. 2, the angular member 28 extends from the bioreactor 10 for directing the flow of fluids out of the bioreactor in a desired direction. The angular member 28 as shown in the figures generally makes a right angle with a straight section 30 of the hollow tubular member 22. The angular member 28, however, can be at any suitable angle with respect to the straight or vertical section 30 of the hollow tubular member 22.

When used to remove fluids from the bioreactor 10, the filter apparatus should have a length sufficient such that the second end 26 of the hollow tubular member 22 resides adjacent to the bottom surface of the bioreactor 10. In this regard, the straight section 30 of the filter apparatus 20 generally has a length greater than the length (or depth) of the bioreactor 10. For instance, the length of the straight section 30 can be greater than about 110%, such as greater than about 120%, such as greater than about 150% of the length of the bioreactor 10. In general, the straight section 30 is less than about 500%, such as less than 300%, such as less than about 200% of the length of the bioreactor 10.

In accordance with the present disclosure, the filter apparatus 20 further includes a filter member 32 positioned at the second end of 26 of the hollow tubular member 22. The filter member 32 is shown in greater detail in FIGS. 4A, 4B and 4C. In accordance with the present disclosure, the filter member 32 has a pore size and a surface area that permits a relatively high flow rate of fluid medium through the filter apparatus 20 while inhibiting the flow of the biological cells through the filter member 32. For example, in one embodiment, the filter member 32 can be made from a porous mesh, such as a stainless steel nonwoven, knitted, or woven material. In one aspect, the filter member 32 can be made from a nonwoven mesh formed from sintered metal fiber, such as stainless steel fiber. Alternatively, the filter member 32 can be made from a polymer material. For instance, in one embodiment, the filter member 32 can be made from a polyamide screen mesh such as a nonwoven material. A polymer mesh, for instance, may be more flexible and less susceptible to damage than a filter element made from a metal. A filter apparatus 20 having a polymer hollow tubular member 22 and filter member 32 may further include a polymer shell (not shown) surrounding the filter member 32.

The pore size of the filter member 32 generally depends upon the size of the cells contained within the bioreactor. The pore size of the filter member 32, for instance, is sufficient to permit flow of a fluid medium without permitting the biological cells from being withdrawn with the fluid medium. The pore size can be uniform over the filter member 32 or nonuniform. In one aspect, the average pore size of the filter member 32 is less than about 12 microns, such as less than about 10 microns, such as less than about 9 microns, such as less than about 8 microns, such as less than about 7 microns, such as less than about 6 microns, such as less than about 5 microns, such as less than about 4 microns. The average pore size of the filter member 32 is generally greater than about 1 micron, such as greater than about 2 microns, such as greater than about 3 microns, such as greater than about 4 microns. Of course, while the pore size has thus far been described as an average pore size, it should be understood that the ranges of pore sizes described above may also reference nominal pore size or absolute pore size. For instance, as an example and as may be known in the art, a nominal pore size of 5 microns would catch about 99% of particles having a pore size of greater than 5 microns. Conversely, an absolute pore size of 5 microns would indicate that all of the pores have a size of no greater than 5 microns, such that all, or substantially all, particles having a size of greater than 5 microns would be caught by the filter. Thus, in one aspect, the pore size of the filter member 32 according to the present disclosure has an absolute pore size according to the above ranges.

The filter member 32 includes an exterior surface that is in direct contact with the fluid medium contained within the bioreactor and an opposite interior surface. The pore size of the filter member 32 on the exterior surface can be different than the pore size of the filter member 32 on the interior surface. In one aspect, for instance, the pore size on the interior surface can be larger than the pore size on the exterior surface. For example, the absolute pore size on the exterior surface can be from about 1 micron to about 9 microns, while the absolute pore size on the interior surface of the filter member 32 can be from about 4 microns to about 20 micron, such as from about 6 microns to about 15 microns. For example, the absolute pore size on the interior surface can be at least about 20% greater, such as at least about 40% greater, such as at least about 60% greater, such as at least 80% greater than the absolute pore size on the exterior surface. In this manner, the pores that run through the filter member 32 can have a funnel-like shape which may allow for greater flow rates through the filter member and may prevent fouling and blockages.

As shown, in FIGS. 4A, 4B and 4C, the filter member 32 is attached to the second end 26 of the hollow tubular member 22. For instance, in the embodiment illustrated, the filter member 32 completely surrounds and encloses the opening located at the second end 26 of the hollow tubular member 22. The filter member 32 can be attached to the hollow tubular member 22 using any suitable method or technique. For instance, the filter member 32 can be welded to the hollow tubular member 22, can be adhered to the hollow tubular member 22 or can be mechanically attached to the hollow tubular member. In one particular embodiment, for instance, the filter member 32 can be resin welded to the hollow tubular member 22.

As shown in FIGS. 4A, 4B and 4C, the filter member 32 has a length that is designed to optimize the surface area and the enclosed volume for ensuring that the filter member 32 can sustain a desired flow rate. The size of the enclosed volume 34 can depend upon the flow requirements of the system and can be proportional to the cross-sectional area of the opening of the second end 26. For instance, the enclosed volume 34 can be of a size sufficient to allow sufficient fluid flow through the filter member and into the hollow tubular member 22 that may be desired for a particular application. The enclosed volume 34, for instance, increases the surface area of the filter member 32 and thus provides more area for fluids to enter the filter member and allows for greater flow rates through the hollow tubular member 22.

For instance, in one embodiment, the ratio between the cross-sectional area of the opening at the second end 26 to the surface area of the filter member 32 can be greater than about 1:5, such as greater than about 1:10, such as greater than about 1:15, such as greater than about 1:20, such as greater than about 1:25, such as greater than about 1:30, such as greater than about 1:35, such as greater than about 1:40. The ratio between the cross-sectional area of the opening of the second end 26 and the surface area 34 of the filter member 32 can generally be less than about 1:1000, such as less than about 1:500, such as less than about 1:200, such as less than about 1:150, such as less than about 1:100, such as less than about 1:80. For example, when the second opening of the second end 26 has a diameter of from about 2 mm to about 20 mm, the filter member 32 can have a length L of generally greater than about 20 mm, such as greater than about 50 mm, such as greater than about 100 mm, such as greater than about 500 mm, and generally less than about 1000 mm, such as less than about 500 mm, such as less than about 200 mm.

The surface area of the exterior surface of the filter member 32 is generally greater than about 0.5 in². For example, the surface area of the filter member 32 can be greater than about 1 in², such as greater than about 1.5 in² such as greater than about 2 in², such as greater than about 2.5 in², such as greater than about 3 in², such as greater than about 3.5 in², such as greater than about 4 in², such as greater than about 5 in². The surface area is generally less than about 100 in², such as less than 10 in².

In the embodiment illustrated in FIG. 4A, the filter member 32 has an elongated shape that terminates at a sloped end 38. It should be understood, however, that the filter member 32 can have any suitable shape. The shape of the filter member 32, for instance, may depend upon a shape that maximizes surface area while being capable of being conveniently placed in the bioreactor 10. It should be understood, however, that the length of the filter member 32 can be much larger than desired above. For example, almost the entire tubular member can be made from the filter member 32.

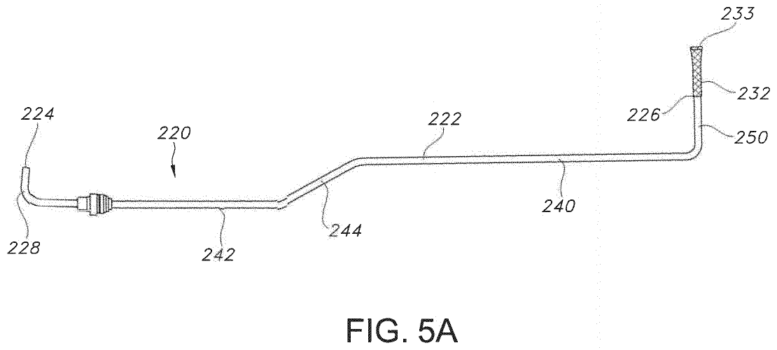

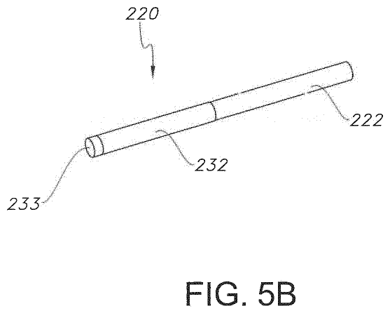

For example, an alternative shape for the filter member is illustrated in FIGS. 5A and 5B. Referring to FIGS. 5A and 5B, the filter apparatus 220 is illustrated including a hollow tubular member 222 connected to a filter member 232. In this embodiment, the filter member 232 has a nonporous end 233. The closed end 233 may protect the filter member 232 from damage when being inserted or removed from a bioreactor.

In accordance with the present disclosure, the cross-sectional area of the hollow tubular member 22, the enclosed volume 34 of the filter member 32, and the pore size of the filter member 32 are all selected so as to optimize flow rates. In particular, the filter apparatus 20 of the present disclosure is designed to allow for relatively high flow rates out of the bioreactor 10. In one embodiment, for instance, the flow rate through the filter apparatus 20 can depend upon the volume of the bioreactor 10. For example, the filter apparatus 20 can be designed to withdraw greater than about 40% of the volume of the bioreactor, such as greater than about 50% of the volume of the bioreactor, such as greater than about 60% of the volume of the bioreactor, such as greater than about 70% of the volume of the bioreactor, such as greater than about 80% of the volume of the bioreactor, such as greater than about 100% of the volume of the bioreactor, such as greater than about 110% of the volume of the bioreactor, such as greater than about 120% of the volume of the bioreactor, such as greater than about 130% of the volume of the bioreactor, such as greater than about 140% of the volume of the bioreactor, such as greater than about 150% of the volume of the bioreactor per day (24 hours). In general, the flow rate through the filter apparatus 20 is generally less than about 500% of the volume of the bioreactor per day, such as less than about 200% of the bioreactor volume per day.

In one particular example, the filter apparatus 20 is designed to withdraw greater than about 0.5 L of fluid per day, such as greater than about 1 L of fluid per day, such as greater than about 2 L of fluid per day, such as greater than about 5 L of fluid per day, such as greater than about 10 L of fluid per day, such as greater than about 20 L of fluid per day, such as greater than about 30 L of fluid per day, such as greater than about 40 L of fluid per day, and generally less than about 100 L per fluid per day out of the bioreactor 10. The flow rate through the filter apparatus 20, for instance, can be greater than about 10 mL/min, such as greater than about 15 mL/min, such as greater than about 20 mL/min, such as greater than about 30 mL/min, such as greater than about 40 mL/min, such as greater than about 50 mL/min, such as greater than about 100 mL/min, such as greater than about 200 mL/min, and generally less than about 2 liters per minute, such as less than about 1 liter per min.

The embodiment of the filter apparatus 20 as shown in FIG. 2 includes a straight or vertical section 30 that is intended to be inserted into the bioreactor 10. Once inserted into the bioreactor 10, the straight or vertical section 30 remains substantially parallel with a vertical axis of the bioreactor and/or with the rotatable shaft 14. Thus, the straight or vertical section 30 has a length that is at least as long as the length or depth of the bioreactor 10. In one embodiment, however, the straight or vertical section 30 may interfere with the impeller 16 contained within the bioreactor 10. Thus, in other embodiments, the shape of the filter apparatus 20 can be altered for providing a better fit within the bioreactor.

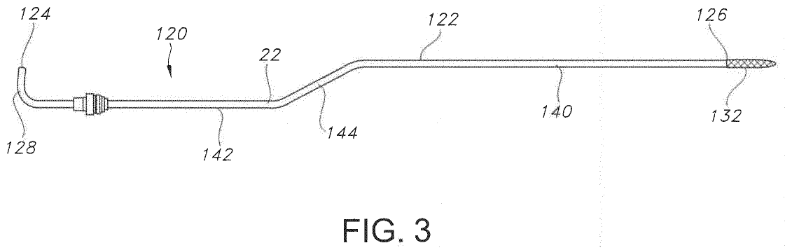

For example, referring to FIG. 3, another embodiment of a filter apparatus 120 is shown. The filter apparatus 120 includes a hollow tubular member 122 including a first end 124 and a second and opposite end 126. Attached to the second end 126 is a filter member 132 made in accordance with the present disclosure. The hollow tubular member 122 further includes an angular member 128 positioned at the first end 124.

In the embodiment illustrated in FIG. 3, the filter apparatus 120 includes a first straight section 140, a second straight section 142, and an angular section 144. The angular section 144 is positioned in between the first straight section 140 and the second straight section 142. As shown in FIG. 1, the angular section 144 can be included in the hollow tubular member 22 in order to prevent the filter apparatus 120 from interfering with an impeller 16 contained within the bioreactor 10. In particular, the angular section 144 positions the second end 126 of the hollow tubular member 122 adjacent to the wall of the bioreactor 10. In one embodiment, the angular section 144 can form an angle with the first straight section 140 of from about 10° to about 80°, such as from about 25° to about 45°. For example, the angle between the angular section 144 and the first straight section 140 can generally be greater than about 20°, such as greater than about 30°, such as greater than about 40°, and generally less than about 60°, such as less than about 50°. Similarly, the angle between the angular section 144 and the second straight section 142 can be from about 10° to about 80°, such as from about 25° to about 45°.

The length of the straight sections 140 and 142 and the length of the angular section 144 can also vary depending upon the geometry of the bioreactor 10 and various other factors. In one embodiment, for instance, the angular section 144 can be greater than about 5%, such as greater than about 10%, such as greater than about 15%, such as greater than about 20%, and generally less than about 50%, such as less than about 40%, such as less than about 30%, such as less than about 20%, of the total length of the first straight section 140, the second straight section 142, and the angular section 144 taken together.

Referring to FIG. 5A, still another embodiment of a filter apparatus 220 made in accordance with the present disclosure is shown. The filter apparatus 220 includes the hollow tubular member 222 including a first end 224 and a second and opposite end 226. The filter member 232 is attached to the second end 226 of the hollow tubular member 222. The hollow tubular member 222 includes a first straight section 250, a second straight second 242, and an angular section 244 positioned in between the first straight section 240 and the second straight section 242. The filter apparatus 220 further includes a first angular member 228 positioned at the first end 224 of the hollow tubular member 222.

In the embodiment illustrated in FIG. 5A, the filter apparatus 220 further includes a second angular member 250 positioned at the second end 226 of the hollow tubular member 222. The second angular member 250 is for positioning the filter member 232 adjacent to the bottom of the bioreactor 10. For instance, the second angular member 250 can form an angle with the first straight section 240 of generally greater than about 40°, such as greater than about 50°, such as greater than about 60°, such as greater than about 70°, such as greater than about 80° and generally less than about 120°, such as less than about 100°. For instance, as shown in FIG. 5A, in one embodiment, the second angular member 250 forms a right angle with the first straight section 240 of the hollow tubular member 222. In this manner, the filter apparatus 220 can be placed in a bioreactor for avoiding interference with an impeller. The second angular member 250, on the other hand, allows for the filter member 232 to extend along the bottom of the bioreactor towards the center of the bioreactor or towards the wall of the bioreactor depending upon the particular application. Thus, the second angular member 250 can have a length suitable to place the filter member 232 at a desired location. The length of the second angular member 250, for instance, in one embodiment, can be generally greater than about 20 mm, such as greater than about 30 mm, such as greater than about 40 mm, such as greater than about 50 mm, such as greater than about 60 mm, such as greater than about 70 mm, such as greater than about 80 mm, such as greater than about 90 mm, such as greater than about 100 mm and generally less than about 500 mm, such as less than about 300 mm, such as less than about 200 mm, such as less than about 180 mm, such as less than about 160 mm, such as less than about 140 mm. The length of the second angular member 250, however, can depend upon the size and volume of the bioreactor 10. Thus, the length can be greater than or less than the dimensions provided above.

Referring to FIG. 6, yet another embodiment of a bioreactor system made in accordance with the present disclosure is shown. The bioreactor system includes a bioreactor 310 having a port 318 located on a side wall of the bioreactor. The bioreactor system further includes a filter apparatus 320 having a hollow tubular member 322 and a filter member 332. The filter apparatus 320 can be inserted into the port 318. The filter member 332 is similar to that as shown in greater detail in FIGS. 4A, 4B and 4C. The filter apparatus 320 may minimize the amount of space occupied in a bioreactor, which in some embodiments may allow the filter member 332 to include a longer mesh having a greater surface area. Allowing filter apparatus 320 access into the bioreactor 310 at the bottom side wall reduces the overall amount of material penetrating into the bioreactor 310, as shown in FIG. 6, compared to embodiments of the filter apparatus that are inserted through a port in the top of the bioreactor, for example as shown in FIG. 1.

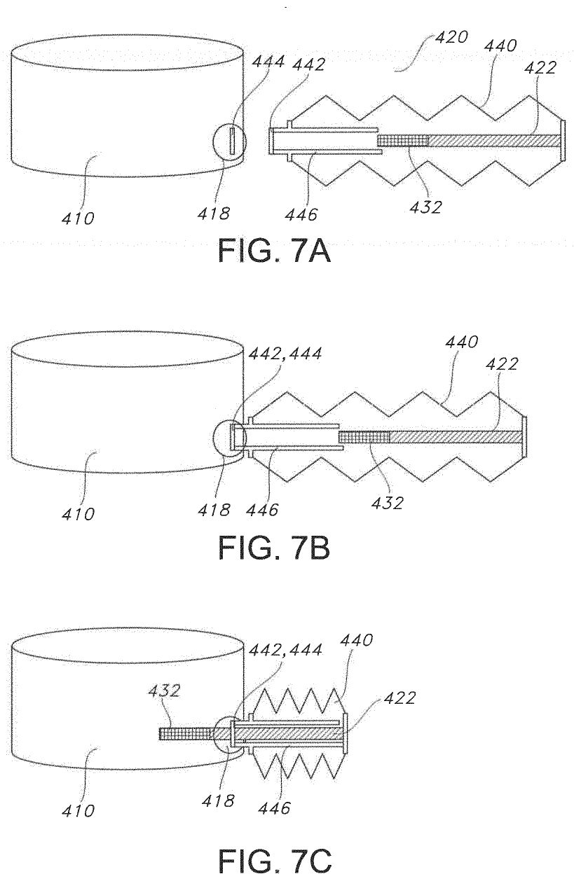

Referring now to FIGS. 7A to 7C, an additional embodiment of a bioreactor system made in accordance with the present disclosure is shown. The bioreactor system includes a bioreactor 410 having a port 418 located on a lower side wall of the bioreactor. The embodiment of FIGS. 7A to 7C further includes a filter apparatus 420 having a hollow tubular member 422 and a filter member 432.

In the embodiment shown in FIGS. 7A to 7C, the filter apparatus 420 further includes a collapsible bellows structure 440 for completely closed, sterile entry. The bellows 420 may be plastic. The hollow tubular structure 422 and the filter member 432 are completely encased in the bellows 440. The bellows 440 forms an enclosed environment that can be sterilized for containing the hollow tubular structure 422 and the filter member 432. The filter apparatus 420 further includes a rigid tunnel 446 within the bellows 440 leading to a sterile connection port 442. The sterile connection port 442 may be any commercially available sterile connection port that is compatible with the bioreactor 410. For example, the sterile connection port may be a Kleenpak™ Sterile Connector manufactured by Pall Biotech, an Opta® sterile connector manufactured by Sartorius, a ReadyMate single-use connector manufactured by GE Healthcare Life Sciences, or other commercially available sterile connector. The bioreactor 410 includes a matching sterile connector 444 in the port 418 on the bioreactor wall.

As shown in FIG. 7B, the sterile connections 442 and 444 of the filter apparatus 420 and bioreactor 410 are first connected to each other. A seal is formed between the sterile connections 442 and 444. Then, as shown in FIG. 7C, an opening is formed between the sterile connections 442 and 444. The bellows 440 can then be collapsed and the hollow tubular member 422 can be pushed through into the bioreactor 410, extending the filter member 432 into the bioreactor 410. The bellows 440 is collapsed when the hollow tubular member 422 and filter member 432 are pushed into the bioreactor.

Referring to FIGS. 8A and 8B, still another embodiment of a bioreactor system made in accordance with the present disclosure is shown. The bioreactor system includes a bioreactor 510 having a cone-shaped filter apparatus 520. The filter member 532 of the filter apparatus 520 is formed as a mesh patch on the wall 511 of the bioreactor 510. The mesh patch may be located on a side wall 511 of the bioreactor 510 as shown in FIG. 8B. The filter apparatus 520 has an enclosed volume 534 formed by a cone 536 that leads from the filter member 532 to an outlet hollow tubular member 522. In some embodiments, the cone 536 may be flexible.

Referring to FIG. 9, an additional embodiment of the bioreactor system made in accordance with the present disclosure is shown. The bioreactor system includes a bioreactor 610 having a cone-shaped filter apparatus 620 that can serve as a filtered drain for the bioreactor 610. The filter member 632 of the filter apparatus 620 is formed as a mesh patch on the bottom wall of the bioreactor 610. The filter apparatus 620 has an enclosed volume 634 formed by a cone 636 that leads from the filter member 632 to an outlet hollow tubular member 622. In some embodiments, the cone 636 may be flexible. The design of the embodiment shown in FIG. 9 allows the maximum amount of liquid to be drained out of the bioreactor.

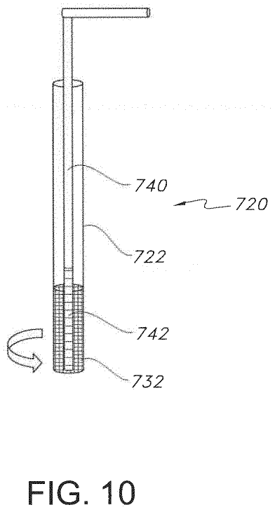

Referring to FIG. 10, another embodiment of a filter apparatus 720 made in accordance with the present disclosure is shown. In this embodiment, the filter apparatus 720 includes a hollow tubular member 722 attached to a filter member 732 made in accordance with the present disclosure and as described above. In this embodiment, however, the filter apparatus 720 includes an inner hollow tube 740 that includes a flow permitting section 742. During operation, as shown by the arrow, the outer tubular member 722 is attached to a motor and rotated. Thus, the outer tubular member 722 and the filter member 732 rotate while the inner tubular member 740 remains stationary. Rotating the outer tubular member 722 can prevent fouling and blockages.

Although the filter apparatus of the present disclosure is generally resistant to fouling and other blockages, various methods and techniques can also be used to prevent or destroy flow blockages. For example, in one aspect, the filter apparatus can be operated periodically in a back flush mode. For example, when withdrawing fluid medium from the bioreactor, the flow of the fluid medium can be reversed at periodic intervals. For instance, in one aspect, flow through the filter apparatus can be reversed at periodic time intervals of from about 30 minutes to about 4 hours, such as at periodic time intervals of from about 45 minutes to about 90 minutes. At the predetermined time interval, flow through the filter apparatus can be reversed for a short amount of time, such as for an amount of time less than about 10 minutes, such as less than 8 minutes, such as less than 5 minutes, and generally for a time greater than about 2 seconds. In this manner, forward flow can occur greater than about 80% of the time, such as greater than about 85% of the time, such as greater than about 90% of the time, such as greater than about 95% of the time, while reverse flow occurs less than 15% of the time, such as less than about 10% of the time, such as less than about 5% of the time during which the filter apparatus is in operation. Periodically reserving flow fluid through the apparatus can remove any matter or debris that has accumulated on the outside surface of the filter member.

The filter apparatus of the present disclosure and particularly in combination with a stirred tank bioreactor can be used in numerous different processes for improving the viability of a biological cell population and/or purifying or otherwise harvesting biological cells. In one application, for instance, a bioreactor can first be filled with a fluid medium, such as a growth medium containing a food source for biological cells. Prior to adding biological cells into the bioreactor, various parameter monitoring devices associated with the bioreactor can be calibrated. For instance, the bioreactor can be placed in association with a probe for measuring dissolved oxygen, pH, temperature, carbon dioxide, and/or oxygen. In addition, the bioreactor can be placed in fluid communication with an air source, a nitrogen gas source, an oxygen gas source, and/or a carbon dioxide gas source. The bioreactor can include an impellor that can be used to agitate or mix the fluid medium. For example, the impeller can rotate at a speed of from about 40 rpm to about 100 rpm, such as from about 85 rpm to about 93 rpm.

Once the pH/dissolved oxygen and/or temperature probes have been calibrated and pH, dissolved oxygen and temperature have stabilized within the bioreactor, the bioreactor can be inoculated with biological cells. In accordance with the present disclosure, any suitable biological cell can be added to the bioreactor, such as mammalian cells. For example, the process and system of the present disclosure is particularly well suited for receiving therapeutic cells including T-cells and NK cells.

The source of the biological cells for inoculating the bioreactor can vary. In one aspect, the biological cells may be obtained from cryogenic bags that need to be thawed and diluted prior to inoculation. When inoculating with T-cells, the proportion of PBMCs that are T-cells can be empirically determined which can be used to estimate later yields.

The cell density of the biological cells during inoculation can vary depending upon the type of cells, the type of bioreactor, and various other factors. In general, the initial cell density within the bioreactor when expanding the cell population is less than about 4×10⁶ cells/mL, such as less than about 2×10⁶ cells/mL, such as less than about 1.5×10⁶ cells/mL, such as less than about 1×10⁶ cells/mL, such as less than about 0.7×10⁶ cells/mL, such as less than about 0.5×10⁶ cells/mL, such as less than about 0.3×10⁶ cells/mL, and generally greater than about 1×10³ cells/mL, such as greater than 1×10⁴ cells/m/L, such as greater than 1×10⁵ cells/mL.

During inoculation, the agitation rates within the bioreactor may be reduced. For instance, the impeller can be rotated at a rate of less than about 79 rpm, such as less than about 65 rpm, and generally greater than about 40 rpm.

The inoculated cells are contained in the bioreactor in an unsupported state suspended in a liquid medium, such as a growth medium containing a food source, such as glucose. In certain embodiments, the inoculated cells need to be activated in order to for cellular expansion to take place. For instance, T-cell activation occurs after activation of the TCR complex and co-stimulation of CD28 by CD80 or CD86. Stimulation can occur that is either antigen dependent or antigen independent. Antigen dependent activation, for instance, expands only antigen specific T-cells whereas antigen independent activation expands all T-cells in the biological cell population.

In one aspect, for unsupported T-cells, activation is initiated by adding to the bioreactor soluble tetrameric antibody complexes that bind CD3 and CD28 cell surface ligands. Addition of the antibody results in the crosslinking of CD3 and CD28 cell surface ligands, thereby providing the required primary and co-signals for T-cell activation. One commercially available activator, for instance, is sold under the name IMMUNOCULT human CD3/CD28 T-cell activator. The activator can be added to the bioreactor using any suitable method, such as by using a sterile syringe.

After the cells have been inoculated and activated, cell growth is promoted within the bioreactor. In one aspect, the bioreactor operates in batch mode for a predetermined period of time, such as for greater than about 1 day, such as greater than about 2 days, such as greater than about 3 days, such as greater than about 4 days, such as greater than about 5 days, and generally less than about 9 days. After a specific period of time, for instance, perfusion mode is activated using the filter apparatus of the present disclosure. Perfusion mode can be initiated, for instance, on day 5, on day 6, on day 7, or on day 8. In one aspect, perfusion mode is activated when a certain cell density is reached. For instance, perfusion mode can be initiated after the cell density has exceeded 1×10⁶ cells/mL, such as greater than about 1.5×10⁶ cells/mL, such as greater than about 1.8×10⁶ cells/mL, such as greater than about 2×10⁶ cells/mL, and generally less than about 5×10⁶ cells/mL.

During batch mode, the agitation rate can be increased to account for higher media volume within the bioreactor. For example, the impeller can be rotated at a speed of greater than about 80 rpm, such as greater than about 85 rpm, such as greater than about 95 rpm, and generally less than about 105 rpm.

During perfusion mode, the fluid medium with the bioreactor is removed as new media is added to the bioreactor for replenishing the media that is withdrawn. During perfusion mode, greater than abut 30% of the fluid volume in the bioreactor, such as greater than about 40% of the fluid volume in the bioreactor, such as greater than about 45% of the fluid volume in the bioreactor is removed and replaced each 24 hours. In general, the perfusion rate is less than 100%, such as less than about 75%, such as less than about 60% of the total medium volume in the bioreactor per day.

In one aspect, the mass or weight of the bioreactor can be periodically or continuously monitored to ensure that the fluid medium volume contained in the bioreactor does not vary by more than about 10%, such as by more than about 5% during the cell expansion process.

The expansion of biological cells according to the above process has been found to dramatically improve not only product quality but also permit the control of various parameters and metabolites in the fluid medium in an automated manner that is completely scalable. For example, remarkably high cell densities can be achieved according to the process of the present disclosure within short periods of time, such as within periods of time of less than about 15 days, such as less than about 14 days, such as less than about 13 days, such as less than about 12 days. Cell densities can be achieved that are greater than about 1×10⁷ cells/mL, such as greater than about 1.2×10⁷ cells/mL, such as greater than about 1.5×10⁷ cells/mL, such as greater than about 2×10⁷ cells/mL. In addition, the viability of the cells can be greater than about 90%, such as greater than about 92%, such as greater than about 95%, such as greater than about 96%.

During the process, glucose levels within the fluid medium in the bioreactor can be controlled to desired levels. For instance, the glucose levels can stay above 4 g/L, such as greater than about 4.5 g/L, such as greater than about 5 g/L, and generally less than about 8 g/L. Lactate levels, on the other hand, can be maintained at relatively low levels. Lactate levels during the process can be below about 1.5 g/L, such as less than about 1.3 g/L, such as less than about 1 g/L. Ammonia levels can also remain relatively low during the process. Ammonia levels can be below about 3 mmol/L.

After a defined period of time or after a cell density is reached, the biological cell population within the bioreactor can be harvested, washed, purified or subjected to various other processes. In one aspect, the biological cell population can be broken down into smaller quantities, optionally concentrated, and fed to cryogenic bags for cryogenic storage. For example, the cryogenic bags can be first frozen and then moved to a liquid nitrogen atmosphere for long term storage.

In addition to washing the biological cell population, in some applications, the biological cell population may include multiple cell types. For example, the biological cell population can include a first type of cell that may be desired and a second type of cell that may be undesired. In accordance with the present disclosure, the different cell types can be easily and efficiently separated from each other.

For example, when producing CAR T-cells, such as allogeneic CAR T-cells, the resulting biological cell population may contain TCR+ cells. The TCR+ cells, in one application, should be separated from the CAR T-cells. The TCR+ cells, for instance, may have a negative effect on patients and lead to graft versus host disease. Similarly, when producing NK cells, the resulting biological cell population may contain CD3+ T-cells that should also be separated and removed from the NK cells. The CD3+ T-cells can also produce undesirable side reactions in patients, such as graft versus host disease. In addition, in some applications, the resulting expanded biological cell population can also contain various cell subsets. For example, a T-cell population may contain CD4+ cells and CD8+ cells. NK cell populations, on the other hand, can contain CD16+ cells. In some applications, it may also be desirable to separate the different cell subsets identified above prior to administering a desired cell type to a patient.

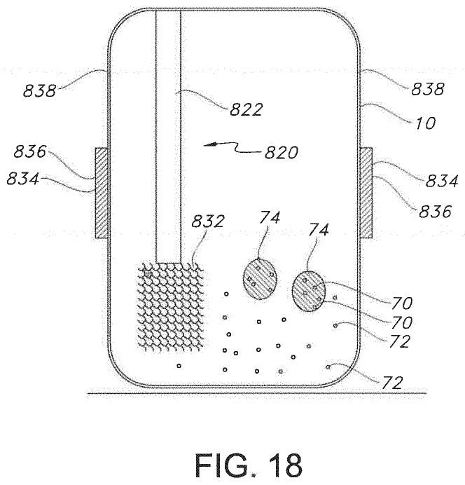

Thus, in accordance with the present disclosure, after a biological cell population has been expanded as described above to a desired cell density, in some embodiments, it may be desirable to separate out different cell types that may be contained within the cell population. One method for separating different cell types in a biological cell population in accordance with the present disclosure is illustrated in FIG. 18. As shown in FIG. 18, a bioreactor 10 contains a biological cell population at a desired cell density. The biological cell population includes different types of cells. In FIG. 18, for instance, the biological cell population includes a first cell type 70 and a second cell type 72. The cell population is contained within the bioreactor 10 within a fluid medium.

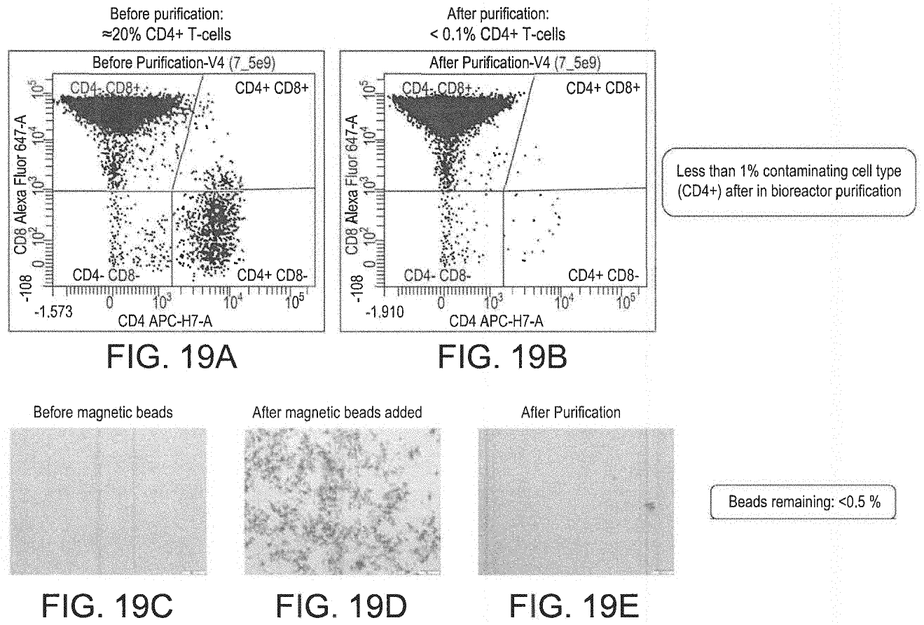

In order to separate the first cells 70 from the second cells 72, microcarriers 74 are added to the bioreactor 10. The microcarriers 74 are made from a material that causes the first cells 70 to attach and bind to the surface of the microcarriers. The second cells 72, however, do not attach and bind to the microcarriers 74. For instance, in some aspects, as shown in FIG. 19, the microcarriers can be magnetic beads, such as paramagnetic beads, which can include one or more surface coatings or functionalizations. Furthermore, it should be understood that separating first cells from second cells can include positive selection or negative selection as understood in the art.

The microcarriers 74, including magnetic bead microcarriers, for instance, can comprise antibody-coupled beads. The antibody present on the beads binds to one of the cell types while not binding to the other cell types. The microcarrier 74 can have any suitable particle size, such as generally greater than about 0.5 microns, such as greater than about 1 micron, such as greater than about, such as greater than about 2 microns, such as greater than about 3 microns, such as greater than about 4 microns, such as greater than about 5 microns, such as greater than about 6 microns, such as greater than about 7 microns, such as greater than about 8 microns, such as greater than about 15 microns, such as greater than about 50 microns, such as greater than about 60 microns, such as greater than about 70 microns, such as greater than about 100 microns, and generally less than about 500 microns, such as less than about 200 microns, such as less than about 175 microns, such as less than about 150 microns, such as less than about 125 microns, such as less than about 100 microns, such as less than about 75 microns, such as less than about 50 microns, such as less than about 25 microns, such as less than about 15 microns, or any ranges or values therebetween.

As shown in FIG. 18, in order to separate the first cells 70 from the second cells 72 after the microcarriers 74 have been added, a filter apparatus 820 can be inserted into the bioreactor 10. The filter apparatus 820 is similar to the filter apparatus as described above with respect to FIGS. 2-10. As shown, for instance, the filter apparatus 820 includes a hollow tubular member 822 in fluid communication with a filter member 832. In this embodiment, however, the filter member 832 has a larger pore size that allows for the passage of the second cells 72 while preventing flow of the microcarriers 74 through the filter member 832.

For example, the filter apparatus 820 can be similar to the filter apparatus described and disclosed in U.S. Patent Publication No. 2019/0136173, which is incorporated herein by reference. The filter member 832, for instance, can have an absolute pore size of generally greater than about 5 microns, such as greater than about 10 microns, such as greater than about 20 microns, such as greater than about 60 microns, such as greater than about 70 microns, such as greater than about 80 microns, such as greater than about 90 microns. The absolute pore size of the filter member 832 is generally less than about 150 microns, such as less than about 130 microns, such as less than about 120 microns, such as less than about 110 microns. The filter apparatus 820 can rapidly and efficiently separate the first cells 70 from the second cells 72.

Additionally or alternatively, in an aspect where the microcarriers are magnetic beads, magnets, including but not limited to Neodymium magnets, can be utilized to further separate cells within the bioreactor. For instance, in one aspect, magnets can be used to separate the cells by introducing the magnets into the bioreactor chamber, such as through hollow tubular member 822, or, additionally or alternatively, mounted to an exterior wall of a bioreactor formed from a material that does not interfere with the magnetic field of the magnets (e.g. referred to as non-reactive herein) in the form of a patch 836 of magnets 834 (shown, for example, by one or more of 836 in FIG. 18).

In one such aspect, the bioreactor can be formed from a rigid or flexible non-reactive plastic, where magnets 834 are mounted on an exterior portion of one or more portions of the side wall 838 of the bioreactor 10. Particularly, the present disclosure has found that a more even magnetic field can be produced that extends along the length of the magnet, instead of being focused near the tip/distal ends when the magnet(s) are applied to one or more portions of the side wall 838 in the form of a patch 836. Thus, greater separation can be obtained using the same size magnet. Furthermore, while shown in the form of a rectangle, it should be understood that the patch may have any shape or cross-section, such as circular, cylindrical, cross (or X-shaped), square, triangular, and the like.

While it should be understood that any pattern may be formed on the exterior wall of the bioreactor using the magnets 834, in one aspect, each patch 836 may have a size sufficient to separate a desired sample size. For instance, in one aspect, a magnet, or magnetic patch having a relatively small surface area (e.g. area in contact with the exterior surface of the bioreactor), such as about 0.4 in² to about 1.5 in², such as about 0.5 in² to about 1.4 in², such as about 0.6 in² to about 1.25 in², such as about 0.7 in² to about 1.2 in², such as about 0.8 in² to about 1.1 in², such as, in one aspect, about 1 in² can be used to separate about 5×10⁹ magnetic beads or more, such as about 5.5×10⁹ magnetic beads or more, such as about 6×10⁹ magnetic beads or more, such as about 7×10⁹ magnetic beads or more, such as about 8×10⁹ magnetic beads or more, such as about 9×10⁹ magnetic beads or more, such as about 10×10⁹ magnetic beads or more, such as at least about 12×10⁹ magnetic beads. Thus, the size of the magnetic patch may be increased or decreased based upon the number of microcarriers to be separated. While the above sizes may be used in reference to a magnet 836 in the form of a patch or placed in to tubular member 822, in one aspect, the above magnet sizes may be in reference to an exterior mounted patch 836.

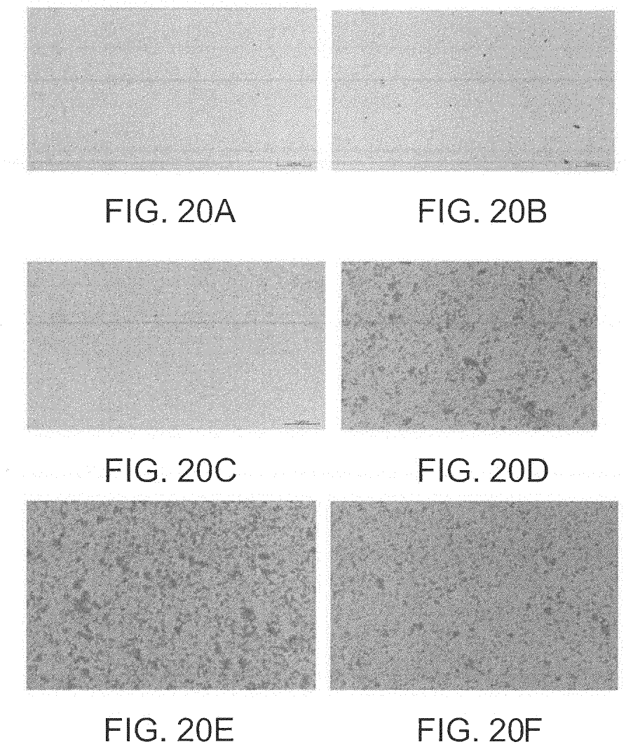

Nonetheless, as shown in FIGS. 19 and 20, the present disclosure has found that by using a patch 836 or placing the magnets 834 into the tubular member 822 in such a manner according to the above sizes, a larger proportion of first cells may be separated from the inoculum, such as about 80% or more of the targeted first cells, such as about 85% or more, such as about 87.5% or more, such as about 90% or more, such as about 92.5% or more, such as about 95% or more, such as about 97.5% or more, such as about 99% or more, or any ranges or values therebetween. For instance, referring to FIG. 19A-C, after purification using magnetic beads, less than 0.1% of CD4+ cells remained in the purified second cell population removed from the bioreactor (e.g. the CD4+ cells remained bound to the microcarriers held in place by one or more magnets 834).

Moreover, as shown in FIGS. 19 C-E, a very small proportion of the beads remain after magnetic separation, such as about 10% or less, such as about 5% or less, such as about 2.5% or less, such as about 2% or less, such as about 1.5% or less, such as about 1% or less, or any ranges or values therebetween This is further shown in FIG. 20 A-E. For instance, FIGS. 20 A-C show a sample that includes magnetic bead microcarriers in the presence of a magnet, where the larger dark circles show the magnetic beads and the small light circles show t-cells). As shown in FIGS. 20A-C, very few magnetic beads remain in the sample, as the magnetic beads are largely captured by the magnets, allowing the unbound cells to be removed from the bioreactor. Additionally, FIGS. 20 D-E, show the same samples 24 hours after removal of the magnets with the magnetic beads re-dispersed in the sample. Thus, it is clear that the magnets disposed on one or more portions of the side of the bioreactor can effectively separate the microcarriers from the inoculum, and also that the magnetic beads can be re-released for cleaning or removal after separation and filtration of the second cells.

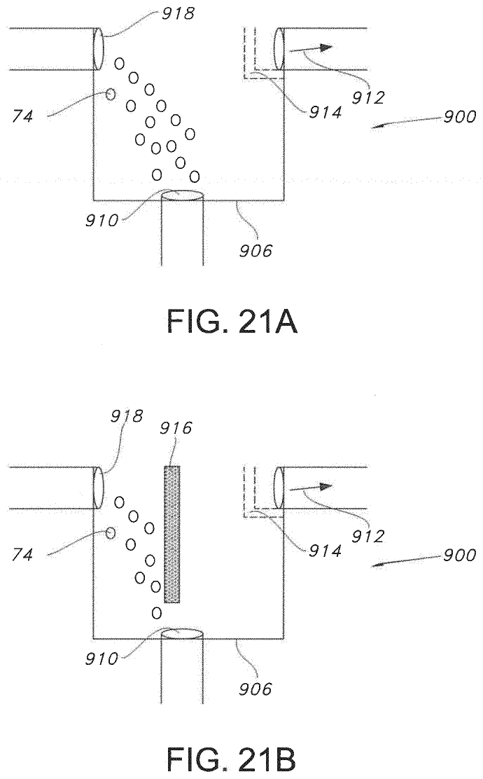

In a further aspect, microcarriers 74 may be allowed to recirculate utilizing an additional or alternative filter according to FIGS. 21 A and B. For instance, as shown in FIG. 21A, the microcarriers 74 and any cells bound thereto may be recirculated through an outlet 910 located on a bottom surface 906 of the external filter 900 which can be in-line with an appropriate bioreactor and any other components discussed herein, which recirculates the microcarriers 74 and any suspended cells back to an appropriate bioreactor. In such an aspect, the conditioned media 912 can be filtered through skimming filter 914, reducing the amount of fouling occurring on the skimming filter 914, as the recirculated material is encouraged to drop away from the skimmer filter 914 due to the density of the microcarriers 74, allowing the conditioned media 912 to proceed through skimming filter 914 without impairment by the microcarriers 74 or any suspended cells. Such a skimming filter 914 can further prevent damage to the microcarriers sometimes experienced during other filtration methods.

Additionally, referring to FIG. 21B, in one aspect, the skimming filter 914 may also include baffle 916. In one aspect, baffle 916 is positioned near inlet 918, which can allow introduction of new media or recirculated media from an appropriate bioreactor. Nonetheless, baffle 916, which can be solid or microporous, deflects microcarriers 908 or suspended cells towards outlet 910 (returning the microcarriers and any suspended cells to an appropriate bioreactor) while allowing the conditioned media 912 to pass through the skimming filter 914. In one aspect, the baffle 916 can be microporous, but it should be understood that the micropores have an average diameter less than the average diameter of the microcarriers.

After filtering, the bioreactor 10 contains the first cells 70 attached to the microcarriers 74. The second cells 72, on the other hand, can be transferred to a new bioreactor. Each cell population (the first cells and the second cells) can then be further purified and washed as desired. If the first cells or second cells are unwanted, either cell population can be discarded as well.

The above process can efficiently and easily separate, for instance, TCR+ cells from CAR T-cells (for instance, by selectively binding to CD3+, CD4+, or and/or separate CD3+ T-cells from NK cells.

In one application, it may be desirable to separate the first cells 70 from the microcarriers 74 after the second cells 72 have been separated from the first cells. Different methods and techniques can be used to separate the first cells 70 from the microcarriers 74. For example, in one embodiment, a separating agent can be added to the bioreactor 10 that causes the cells to separate from the microcarriers. After separation, the filter apparatus 820 can then be used to remove the first cells 70 from bioreactor 10 and separate them from the microcarriers 74.

In an alternative embodiment, the microcarriers 74 can be designed to be dissolvable within the fluid medium contained within the bioreactor 10. For example, the microcarrier 74 can dissolve in the fluid medium or a dissolving agent can be added to the fluid medium for dissolving the microcarriers 74. Once the microcarriers have been dissolved, the first cells 70 remain within the bioreactor 10 in an unsupported state and can be further washed and purified.

Once a desired biological cell population has been optionally isolated and separated from other cells, the cell population can be washed and purified according to methods of the present disclosure using the filter apparatus as shown and described with respect to FIGS. 1-10. In one aspect, for instance, the fluid medium in which the biological cell population is contained is withdrawn from the bioreactor using the filter apparatus of the present disclosure, such as filter apparatus 20. The filter apparatus, for instance, can remove at least about 30%, such as at least about 40%, such as at least about 50%, such as at least about 60%, such as at least about 70%, such as at least about 80% of the volume of the fluid medium within the bioreactor. In order to wash and purify the cells, a buffer medium can then be added to the bioreactor to replace the fluid medium that is withdrawn.

During the above wash process, the biological cell population is purified by removing and separating the fluid medium from the cells and any biological byproducts or other contaminants that may be in the fluid medium. For example, during cell expansion, the biological cells may produce byproducts such as proteins. In addition, serum may also be contained in the fluid medium. Through the process of the present disclosure, such biological byproducts such as proteins and serum can be removed and separated from the cells. The washing process can be conducted multiple times (i.e. multiple cycles) in order to reach a desired purity level. For instance, the cells can be washed such that the resulting cell population contains biological byproducts, such as proteins and serum, in an amount less than 0.1% by weight. For example, the cell population can be washed through the above described method greater than 1 cycle, such as greater than 2 cycles, such as greater than 3 cycles, and generally less than about 10 cycles, such as less than about 8 cycles, such as less than about 6 cycles, such as less than about 5 cycles.

Of particular advantage, the biological cell population can be washed in accordance with the present disclosure in an automated manner that does not require centrifuging or significant amounts of manual labor. In addition, washing can be done very efficiently and quickly. For instance, the fluid medium can be withdrawn from the bioreactor at a flow rate such that at least about 50% of the volume of the fluid medium, such as at least about 60% of the volume of the fluid medium, such as at least about 70% of the volume of the fluid medium can be removed from the bioreactor in less than about 4 hours, such as less than about 3 hours, such as less than about 2 hours, such as even less than about 1 hour.

In addition, the method is completely scalable. For instance, the cell population that is washed according to present disclosure can be contained in smaller bioreactors having volumes of from about 1 to about 10 L, or can occur in larger bioreactors having volumes of from about 5 L to about 75 L.

After the cell population has been washed to a desired level of purity, the cell population is contained within the buffer medium. The resulting product can then be transferred to flexible bag vessels for cryogenic storage. In one embodiment, a cryogenic buffer medium can also be added to the cell population prior to freezing. The cryogenic buffer medium, for instance, can be CRYOSTOR 10 sold by Biolife Solutions. The cryogenic buffer medium can be serum-free and contain an alkyl sulfoxide, such as dimethyl sulfoxide. The dimethyl sulfoxide can be present in the cryogenic buffer medium in an amount greater than about 5% by weight and in an amount less than about 25% by weight.

The cell population for storage can have relatively high purity levels. Because the process is automated, the high purity levels can be maintained from batch to batch without fluctuation. Thus, due to the high purity levels, greater cell densities can be frozen and stored and later delivered to patients. For instance, cell densities within the flexible storage bags can be greater than about 9,000,000 cells per mL, such as greater than about 10,000,000 cells per mL, such as greater than about 12,000,000 cells per mL.

Through the process of the present disclosure, cell densities can be expanded by greater than 200%, including T-cell populations and NK cell populations. Once the cell population has expanded, the cells can be separated from undesired cells to and/or purified using the methods as described above. In addition, processes according to the present disclosure can produce cell populations having greater proportionate amounts of desired phenotypes. Ultimately, a cell population can be achieved that has greater than 90% purity. For instance, cell populations can be achieved having greater than 90% purity of CD4 T-cells and/or CD8 T-cells. In addition, the process can be carried out with less than 10% total cell loss and greater than 90%, such as greater than 95% cell viability. Cell concentrations can be increased by up to 3 times in relation to past processes which reduces the needed volume of diafiltration media. In addition, the final cell product can contain residual serum/protein levels in amounts less than 0.1% by weight. Finally, post cryogenic viability of the cells can be greater than 90% with greater than a 30 fold expansion following reactivation.

The present disclosure may be better understood with reference to the following examples.

### EXAMPLES

### Example No. 1

The following example was conducted in order to test the ability of a filter apparatus made in accordance with the present disclosure to withdraw a liquid medium from a stirred tank bioreactor without also withdrawing or harming biological cells contained in the bioreactor.

The filter apparatus used in the following experiments is similar to the design illustrated in 4A. The filter member had a length of approximately 1.5 in² and the exterior surface of the filter member had a surface area of 0.65 in². The filter member was made from sintered stainless steel fibers and had an absolute pore size of 3-4 microns. The tubular member attached to the filter member had an inside diameter of 6.35 mm.

T-cells were inoculated in a 1 liter bioreactor at a density of 3-6.5×10⁶ cells/mL. The bioreactor was a stirred tank bioreactor. The cell culture was maintained within the bioreactor for 1 day during which fluid medium was withdrawn from the bioreactor using the filter apparatus as described above.

The filter apparatus was operated at 3 different flow rates. The flow rates included a first flow rate at 5 to 10 mL/mins, an intermediate flow rate of 10 to 15 mL/mins and a high flow rate of 20 to 25 mL/mins. Samples of fluid medium withdrawn from the bioreactor were collected and analyzed. At all three flow rates, cell loss within the bioreactor was less than 2%.

In a separate experiment, the filter apparatus was operated at an intermittent perfusion regime (on for 5 minutes every hour) at a low-medium flow rate (5 mL per minute, over 5 minutes=25 mL removed per hour). The initial inoculation density was 0.5×10⁶ cells/mL, with T-cells activated and expanding subsequent to inoculation. Perfusion was initiated 5 days after T-cell inoculation and activation (Culture Day 5). Culture day six (e.g. 24 hours after perfusion was initiated), 522 mL of the fluid medium was successfully perfused out of the bioreactor. At day twelve, 3,650 m/L of the fluid medium was successfully perfused out of the bioreactor. Some clogging was noticed after twelve days of service. During the perfusion mode, cell expansion was observed.

### Example No. 2

In this example, a stirred tank bioreactor operated in batch mode was compared with a stirred tank bioreactor operated according to the present disclosure using the filter apparatus as described in Example No. 1.

For both bioreactors, CD3+ T-cells were isolated from PBMCs and inoculated into 1 liter stirred tank bioreactors. The pH setpoint was less than 7.2. The dissolved oxygen setpoint was greater than 50%. At day 0, IMMUNOCULT CD3/CD28 activator was added. The growth medium added to the bioreactor was XVIVO15, 5% human serum, 25 IU IL-2. Initial media volume was 400 mL and the initial cell density was 220×10⁶ cells/mL.

Each bioreactor was operated for 18 days. 800 mL of new liquid growth medium was added to each bioreactor at day 3. No further changes were made to the batch mode stirred tank bioreactor.

With respect to the bioreactor system made in accordance with the present disclosure, perfusion was started on day 5. During perfusion, approximately one half of the volume of the fluid medium within the bioreactor was withdrawn and replaced. An intermittent perfusion regime was used in which 25 mL of fluid medium was withdrawn every hour over fixed 5 minute intervals. Perfusion lasted 9 days and ended on day 14.

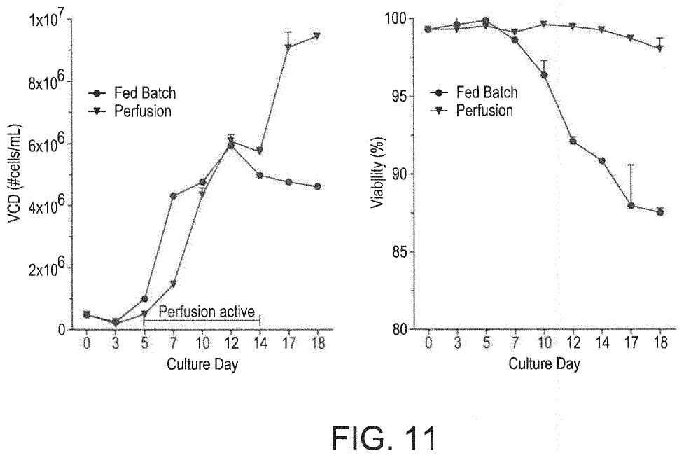

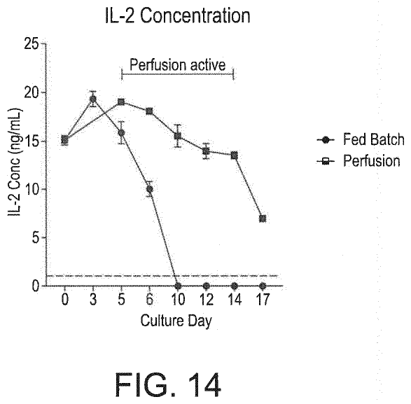

The results are illustrated in FIGS. 11 through 14. FIG. 11 for instance, illustrates the viable cell density and percent viability of both systems. As shown, the bioreactor operated in accordance with the present disclosure display dramatic improvements especially after day 12.

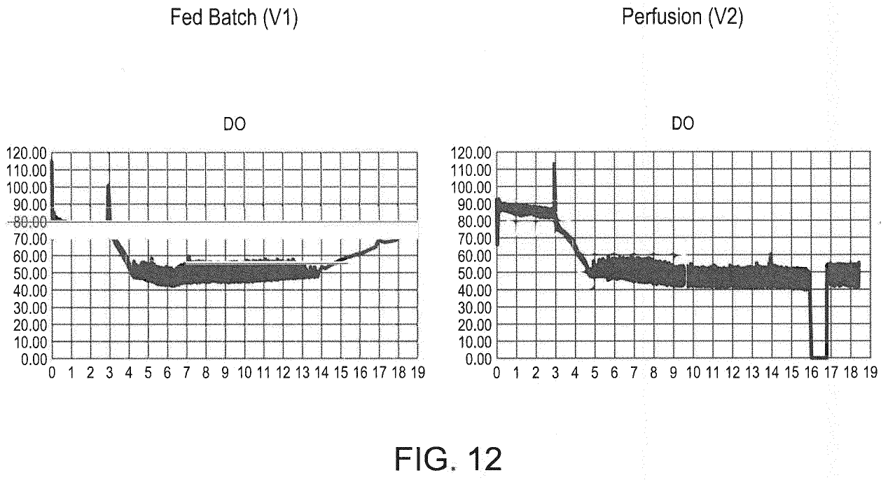

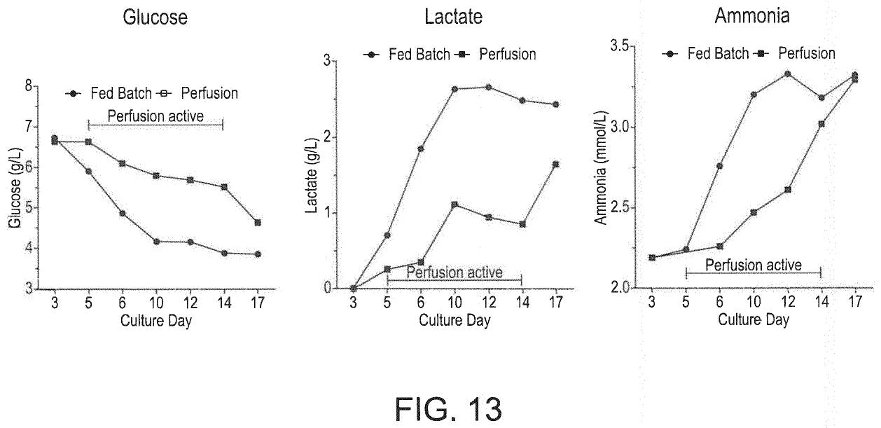

FIGS. 12, 13 and 14 illustrate dissolved oxygen levels, glucose levels, lactate, ammonia levels, and IL-2 concentration over the course of the experiment. As shown in FIG. 13, glucose and lactate levels especially were better controlled using the bioreactor of the present disclosure.

### Example No. 3

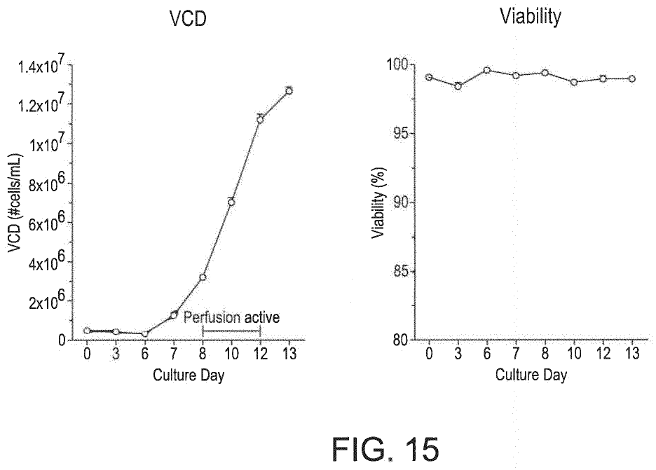

In this example, T-cells were expanded in a stirred tank bioreactor using the filter apparatus of the present disclosure according to the same process as described above with respect to Example No. 2. In this example, however, perfusion was started after the T-cell population exceeded a cell density of 2×10⁶ m/L. This occurred on day 8. The results are illustrated in FIGS. 15-18. As shown in FIG. 15, cell expansion increased dramatically after perfusion was initiated. The cell viability also stayed above 96% during the entire process.

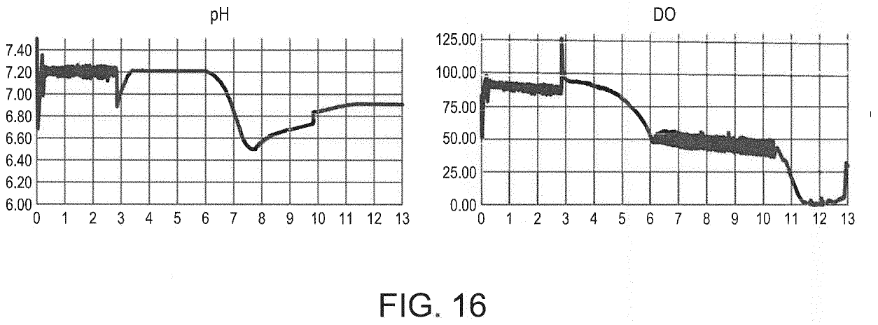

As shown in FIG. 16, dissolved oxygen rapidly decreased to 0 starting on day 10 through day 13. One would normally expect that such a decrease in dissolved oxygen would have a negative effect on cell growth. To the contrary, cell expansion rapidly increased during the same period.

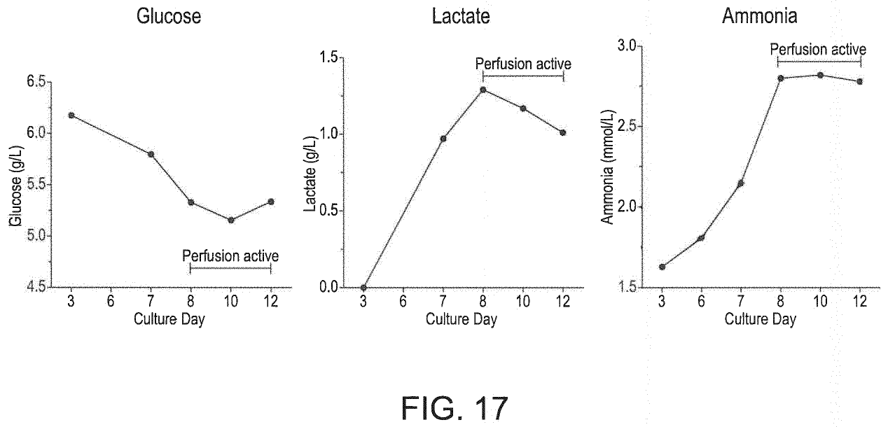

As shown in FIG. 17, glucose, lactate, and ammonia levels were controlled and were maintained at optimal levels during the process.

T-cell phenotypes were also tested after day 13 and compared to a batch stirred tank reactor process. The following results were obtained.

| | Fed Batch STR | Perfusion STR |
|---|---|---|
| Attribute: | (Day 10: Donor 1) | (Day 13: Donor 2) |
| T-cell Purity (CD3+) | 98% | 99% |
| CD4:CD8 ratio | 59%:39% | 60%:36% |
| T_{cm} cells (CD3+ | 88% | 89% |
| CD62L+) | | |
| T_{scm} cells (CD45ra+, | 6.3% | 44% |
| CD62L+) | | |
| Exhaustion Markers | | <5% |
| PD1+ | 1% | 1% |
| CTLA4+ | 1% | 1% |
| Senescence Markers | | <10% |
| KLRG1+ | 1% | 6% |
| CD57+ | 12% | 9% |

As shown above, there was an unexpected and dramatic increase in T_{scm} cells, which is the preferred phenotype. Although unknown, it is believed that the increase in the amount of T_{scm} cells may be due or may occur in conjunction with the lower dissolved oxygen levels.

The devices, facilities and methods described herein are suitable for use in and with culturing any desired cell line including prokaryotic and/or eukaryotic cell lines. Further, in embodiments, the devices, facilities and methods are suitable for culturing suspension cells or anchorage-dependent (adherent) cells and are suitable for production operations configured for production of pharmaceutical and biopharmaceutical products-such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermentor or fermentation unit, or any other reaction vessel and the term “reactor” is used interchangeably with “fermentor.” For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and C02 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBI, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBvl3.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57Bl/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, N.C., USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii), the Geotrichum genus (e.g. Geotrichum fermentans), Hansenula polymorpha, Yarrowia lipolytica, or Schizosaccharomyces pombe, Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In one embodiment, the prokaryotic cell is a Gram-negative cell, such as Salmonella spp. or Escherichia coli, such as e.g., TG1, TG2, W3110, DH1, DHB4, DH5a, HMS 174, HMS174 (DE3), NM533, C600, HB101, JM10⁹, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the protein is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 1111n-hEGF, AE-37, trasnizumab-DM1, Antagonist G, IL-12 (recombinant), PM-02734, IMP-321, rhlGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36](obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-1, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In some embodiments, the polypeptide is adalimumab (HUMIRA), infliximab (REMICADE™), rituximab (RITUXAN™/MAB THERA™) etanercept (ENBREL™), bevacizumab (AVASTIN™), trastuzumab (HERCEPTIN™) pegrilgrastim (NEULASTA™), or any other suitable polypeptide including biosimilars and biobetters.

Other suitable polypeptides are those listed below and in Table 1 of US2016/0097074:

TABLE I

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant dimeric fusion | Amevive ® |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumornab; technetium-99 labeled | CEA-Scan ® |
| alglucerase; modified form of | Ceredase ® |
| beta-glucocerebrosidase | |
| imiglucerase; recombinant form of | Cerezyme ® |
| beta-glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand | Humate-P ® |
| factor complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony- | Leukine ®/Leukine ® |
| stimulating factor | Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ |
| | (formerly LeuTech ®) |
| somatropin [rDNA] | Norditropin ®/Norditropin |
| | Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion | Orencia ™ |
| protein | |
| muromomab-CD3 | Orthoclone OKT3 ® |
| high-molecular weight hyaluronan | Orthovisc ® |
| human chorionic gonadotropin | Ovidrel ® |
| live attenuatedBacillusCalmette-Guerin | Pacis ® |
| abatacept, fully human soluable fusion | Orencia ™ |
| protein | |
| muromomab-CD3 | Orthoclone OKT3 ® |
| high molecular weight hyaluronan | Orthovisc ® |
| human chorionic gonadotropin | Ovidrel ® |
| live attenuatedBacillusCalmette-Guerin | Pacis ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective reversible T-cell | RAPTIVA ™ |
| blocker | |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLARIS (R) |
| Pegvisomant | SOMAVERT ® |
| Palivizumab; recombinantly produced, | Synagis ™ |
| humanized mAb | |
| thyrotropin alfa | Thyrogen ® |
| Tenecteplase | TNKase ™ |
| Natalizumab | TYSABRI ® |
| human immune globulin intravenous | Venogiobulin-S ® |
| 5% and 10% solutions | |
| interferon alfa-n1, lymphoblastoid | Wellferon ® |
| drotrecogin alfa | Xigris ™ |
| Omalizumab; recombinant | Xolair ® |
| DNA-derived humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In embodiments, the polypeptide is a hormone, blood clotting/coagulation factor, cytokine/growth factor, antibody molecule, fusion protein, protein vaccine, or peptide as shown in Table 2.

TABLE 2
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, Norditropin, |
| | somatotropin | NovIVitropin, Nutropin, Omnitrope, Protropin, |
| | Human follicle-stimulating | Siazen, Serostim, Valtropin |
| | hormone (FSH) | Gonal-F, Follistim |
| | Human chorionic gonadotropin | Ovidrel |
| | Lutropin-α | Luveris |
| | Glucagon | GlcaGen |
| | Growth hormone releasing | Geref |
| | hormone (GHRH) | ChiRhoStim (human peptide), SecreFlo |
| | Secretin | (porcine peptide) |
| | Thyroid stimulating hormone | Thyrogen |
| | (TSH), thyrotropin | |
| Blood | Factor VIIa | NovoSeven |
| Clotting/Coagulation | Factor VIII | Bioclate, Helixate, Kogenate, Recombinate, |
| Factors | Factor IX | ReFacto |
| | Antithrombin III (AT-III) | Benefix |
| | Protein C concentrate | Thrombate III |
| | | Ceprotin |
| Cytokine/Growth | Type I alpha-interferon | Infergen |
| factor | Interferon-αn3 (IFNαn3) | Alferon N |
| | Interferon-β1a (rIFN-β) | Avonex, Rebif |
| | Interferon-β1b (rIFN-β) | Betaseron |
| | Interferon-γ1b (IFN γ) | Actimmune |
| | Aldesleukin (interleukin | Proleukin |
| | 2(IL2), epidermal theymocyte | Kepivance |
| | activating factor; ETAF | Regranex |
| | Palifermin (keratinocyte | Anril, Kineret |
| | growth factor; KGF) | |
| | Becaplemin (platelet-derived | |
| | growth factor; PDGF) | |
| | Anakinra (recombinant IL1 | |
| | antagonist) | |
| Antibody molecules | Bevacizumab (VEGFA mAb) | Avastin |
| | Cetuximab (EGFR mAb) | Erbitux |
| | Panitumumab (EGFR mAb) | Vectibix |
| | Alemtuzumab (CD52 mAb) | Campath |
| | Rituximab (CD20 chimeric | Rituxan |
| | Ab) | Herceptin |
| | Trastuzumab (HER2/Neu | Orencia |
| | mAb) | Humira |
| | Abatacept (CTLA Ab/Fc | Enbrel |
| | fusion) | Remicade |
| | Adalimumab (TNFα mAb) | Amevive |
| | Etanercept (TNF receptor/Fc | Raptiva |
| | fusion) | Tysabri |
| | Infliximab (TNFα chimeric | Soliris |
| | mAb) | Orthoclone, OKT3 |
| | Alefacept (CD2 fusion protein) | |
| | Efalizumab (CD11a mAb) | |
| | Natalizumab (integrin α4 | |
| | subunit mAb) | |
| | Eculizumab (C5mAb) | |
| | Muromonab-CD3 | |
| Other: | Insulin | Humulin, Novolin |
| Fusion | Hepatitis B surface antigen | Engerix, Recombivax HB |
| proteins/Protein | (HBsAg) | Gardasil |
| vaccines/Peptides | HPV vaccine | LYMErix |
| | OspA | Rhophylac |
| | Anti-Rhesus(Rh) | Fuzeon |
| | immunoglobulin G | QMONOS |
| | Enfuvirtide | |
| | Spider silk, e.g., fibrion | |

In embodiments, the protein is multispecific protein, e.g., a bispecific antibody as shown in Table 3.

TABLE 3
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant ascites |
| (Removab ®, | Triomab | EpCAM | cells to tumor, Fc | EU | in EpCAM |
| Fresenius Biotech, | | | mediated effector | | positive tumors |
| Trion Pharma, | | | functions | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II and | ALL |
| MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal cancer |
| (Macrogenics) | | | cells to tumor | | |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of NK | | |
| Therapeutics) | | | cells to tumor | | |
| | | | cells | | |
| AFM13 (Affimed | TandAb | CD30, | Retargeting of NK | Phase II | Hodgkin's |
| Therapeutics) | | CD16A | cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded | | cells to tumor | | and |
| Institute) | with BsAb | | | | osteosarcoma |
| pGD2 (Barbara | T cells | CD3, Her2 | Retargeting of T | Phase II | Metastatic breast |
| Ann Karmanos | preloaded | | cells to tumor | | cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T cells | preloaded | | activated T-cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, | Retargeting of T | Phase II | Metastatic |
| Hospital Tübingen) | scFv | MAPG | cells to tumor | | melanoma |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University of | linked to | | protein toxin to | | or lymphoma |
| Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, VEGF | Blockade of 2 | Phase I | Solid tumors |
| (RO5520985, | | A | proangiogenics | | |
| Roche) | | | | | |
| RG7716 (Roche) | CrossMab | Ang2, VEGF | Blockade of 2 | Phase I | Wet AMD |
| | | A | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL-17A | Blockade of 2 | Phase II | Rheumatoid |
| (AbbVie) | | | proinflammatory | | arthritis |
| | | | cytokines | | |
| COVA322 | IgG- | TNF, IL17A | Blockade of 2 | Phase I/II | Plaque psoriasis |
| | fynomer | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, Ablynx) | | | proinflammatory | | arthritis |
| | | | cytokine, binds to | | |
| | | | HSA to increase | | |
| | | | half-life | | |
| ALX-0761 (Merck | Nanobody | IL-17A/F, | Blockade of 2 | Phase I | (Healthy |
| Serono, Ablynx) | | HSA | proinflammatory | | volunteers) |
| | | | cytokines, binds | | |
| | | | to HSA to | | |
| | | | increase half-life | | |
| ALX-0061 | Nanobody | IL-6R, HSA | Blockade of | Phase I/II | Rheumatoid |
| (AbbVie, Ablynx; | | | proinflammatory | | arthritis |
| | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | HSA | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method for purifying a cellular population comprising:
expanding a biological cell population in a fluid medium, the biological cell population comprising biological cells in an unsupported state, the biological cell population being contained in a bioreactor and having a cell density of at least 1×10⁶ cells/mL;
removing and filtering the fluid medium from the bioreactor, the fluid medium being filtered through a filter apparatus comprising a filter member, the filter member having a pore size that inhibits the biological cells from being withdrawn from the bioreactor as the fluid medium is withdrawn; and
adding to the biological cell population a buffer medium.

2. A method as defined in claim 1, wherein the fluid medium contains biological byproducts and wherein the filter member has a pore size that permits passage of the biological byproducts with the fluid medium that is withdrawn.

3. A method as defined in claim 1, wherein greater than about 50% of the volume of the fluid medium is withdrawn and at least partially is replaced with the buffer medium.

4. A method as defined in claim 1, wherein the biological cell population and fluid medium has a volume of from about 1 L to about 10 L.

5. A method as defined in claim 1, wherein the method is repeated from about 2 cycles to about 5 cycles.

6. A method as defined in claim 1, wherein the biological cells comprise T cells or NK cells.

7. A method as defined in claim 1, further comprising the step of dispensing the biological cell population and buffer medium into flexible bag vessels for cryogenic storage.

8. A method as defined in claim 1, wherein the filter member of the filter apparatus has an absolute pore size of from about 1 micron to 9 microns.

9. A method as described in claim 1, a wherein the filter apparatus comprises a hollow tubular member having a first end defining a first opening and a second and opposite end defining a second opening, the filter member being located at the second end of the hollow tubular member, the filter member completely surrounding and enclosing the second opening, the filter member defining an interior surface and an exterior surface.

10. A method as defined in claim 1, wherein the biological cell population comprises at least two different cell types including first cells and second cells and wherein the method further comprises placing one or more microcarriers in the fluid medium with the biological cell population and wherein the first cells bind to the one or more microcarriers but the second cells do not; and
removing and filtering the fluid medium from the bioreactor, the fluid medium being filtered through a second filter apparatus comprising a filter member, the filter member having a pore size that permits passage of the second cells but inhibits passage of the one or more microcarriers for separating the first cells from the second cells, optionally wherein at least a portion of the one or more microcarriers is magnetic.

11. A method as defined in claim 10, wherein at least one Formal of the first cells or the second cells comprise T-cells or NK cells.

12. A method according to claim 1, wherein the filter apparatus is periodically operated in a back flush mode or is configured for continuous perfusion.

13. A filter apparatus suitable for use in bioreactors comprising:
a hollow tubular member for filtering fluid from a bioreactor, the hollow tubular member having a first end defining a first opening and a second and opposite end defining a second opening; and
a filter member located at the second end of the hollow tubular member, the filter member completely surrounding and enclosing the second opening, the filter member defining an interior surface and an exterior surface, the filter member comprising a porous material, the porous material having an absolute pore size of from about 1 micron to about 9 microns.

14. A filter apparatus as defined in claim 13, wherein
the filter member comprises a porous mesh,
the porous material has an absolute pore size of from about 1 micron to about 6 microns,
the filter member comprises a nonwoven mesh formed from sintered metal fiber.

15. A filter apparatus as defined in claim 13, wherein the filter member has a length along an axial direction of the hollow tubular member, the length of the filter member being 1 inch or greater and about 12 inches or less.

16. A filter apparatus as defined in claim 13, wherein the exterior surface of the filter member has a surface area and wherein the surface area is greater than about 0.5 in².

17. A filter apparatus as defined in claim 13, wherein the ratio between the cross-sectional area of the second opening and the surface area of the filter member is from about 1:5 to about 1:200.

18. A filter apparatus as defined in claim 13, wherein the hollow tubular member:
is made from stainless steel or a thermoplastic polymer, or
has a diameter of from about 0.2 inches to about 0.7 inches.

19. A filter apparatus as defined in claim 13, wherein the interior surface of the filter member has an absolute pore size and the exterior surface of the filter member has an absolute pore size, and wherein the absolute pore size of the interior surface is larger than the absolute pore size of the exterior surface, the absolute pore size of the exterior surface being from about 1 micron to about 9 microns.

20. A filter apparatus as defined in claim 13, wherein the hollow tubular member includes a first straight section, a second straight section, and an angled section positioned between the first straight section and the second straight section, the angled section for locating the second opening and filter member at a location in a bioreactor without contacting a rotating impeller.

21. A filter apparatus as defined in claim 13, wherein the hollow tubular member includes an angular member located adjacent to the second end, the hollow tubular member including a straight section that transitions into the angular member, the angular member being at an angle to the straight section of from about 500 to about 90°.

22. A filter apparatus as defined in claim 13, wherein the hollow tubular member and the filter member are movably enclosed in a collapsible bellows, wherein the collapsible bellows includes a sterile connection port on one end for connecting to a matching sterile connection port of the bioreactor.

23. A filter apparatus as defined in claim 13, wherein the filter member includes a mesh patch on a side or bottom wall of a bioreactor, the filter member further including a cone connecting the mesh patch to the hollow tubular member.

24. A method for culturing cell growth comprising:
inoculating biological cells into a bioreactor, the bioreactor containing a fluid medium for cell growth;
perfusing the fluid medium contained in the bioreactor by inserting into the bioreactor a filter apparatus according to claim 13; and
replenishing the fluid medium within the bioreactor in order to promote cell growth.
